(19) United States
(12) Patent Application Publication
(10) Pub. No.: US 2002/0010341 A1
(43) Pub. Date: Jan. 24, 2002

(54) Use of thioamide oxazolidinones for the treatment of bone resorption and osteoporosis

(76) Inventors: Gebre-mariam Mesfin, Portage, MI (US); Richard K. Jensen, Kalamazoo, MI (US)

Correspondence Address: Andrew M. Solomon, Pharmacia & Upjohn Company, Global Intellectual Property, 301 Henrietta Street, Kalamazoo, MI, 49001, US

(21) Appl. No.: 09/836,804

(22) Filed: Apr. 17, 2001

Related U.S. Application Data
(60) Provisional application No. 60/198,688, filed on Apr. 20, 2000.

(51) Int. Cl. C07D 263/20; C07D 261/02; C 07D 3 7/40; A61K 31/365; A61K 31/42; A61K 31/421

(52) U.S. Cl. 548/229; 548/240; 549/318; 514/376; 514/378; 514/471

(57) ABSTRACT

The use of thioamide oxazolidinones for the treatment of bone resorption and osteoporosis is provided.

### BACKGROUND OF THE INVENTION

1. Field of the Invention

The present invention is directed to a new use for known compounds. More specifically, the invention relates to the use of thioamide oxazolidinones for the treatment of bone resorption, osteoporosis and other bone diseases.

2. Technology Description

Bone is not a static tissue. It is subject to constant breakdown and resynthesis in a complex process mediated by osteoblasts, which produce new bone, and osteoblasts, which destroy bone. The activities of these cells are regulated by a large number of cytokines and growth factors, many of which have now been identified and cloned. Mundy has described the current knowledge related to these factors (Mundy, G. R. Clin Orthop 324:24-28, 1996; Mundy, G. R. J Bone Miner Res 8:S505-10, 1993).

Although there is a great deal of information available on the factors which influence the breakdown and resorption of bone, information on growth factors that stimulate the formation of new bone is more limited. Investigators have searched for sources of such activities, and have found that bone tissue itself is a storehouse for factors that have the capacity for stimulating bone cells. Thus, extracts of bovine bone tissue obtained from slaughterhouses contain not only structural proteins which are responsible for maintaining the structural integrity of bone, but also biologically active bone growth factors which can stimulate bone cells to proliferate. Among these latter factors are transforming growth factor beta., the heparin-binding growth factors (e.g., acidic and basic fibroblast growth factor), the insulin-like growth factors (e.g., insulin-like growth factor I and insulin-like growth factor II), and a recently described family of proteins called bone morphogenetic proteins (BMPs). All of these growth factors have effects on other types of cells, as well as on bone cells.

The BMPs are novel factors in the extended transforming growth factor beta. superfamily. They were first identified by Wozncy J. et al. Science (1988) 242:1528-34, using gene cloning techniques, following earlier descriptions characterizing the biological activity in extracts of demineralized bone (Urist M. Science (1965) 150:893-99). Recombinant BMP2 and BMP4 can induce new bone formation when they are infected locally into the subcutaneous tissues of rats (Wozncy J. Molec Reprod Dev (1992) 32: 160-67). These factors are expressed by normal osteoblasts as they differentiate, and have been shown to stimulate osteoblast differentiation and bone nodule formation in vitro as well as bone formation in vivo (Harris S. et al. J. Bone Miner Res (1994) 9:855-63). This latter property suggests potential usefulness as therapeutic agents in diseases that result in bone loss.

The cells that are responsible for forming bone are osteoblasts. As osteoblasts differentiate from precursors to mature bone-forming cells, they express and secrete a number of enzymes and structural proteins of the bone matrix, including Type-i collagen, osteocalcin, osteopontin and alkaline phosphatase (Stein G. et al Curr Opin Cell Biol (1990) 2:1018-27; Harris S. et al. (1994), supra). They also synthesize a number of growth regulatory peptides that are stored in the bone matrix, and are presumably responsible for normal bone formation. These growth regulatory peptides include the BMPs (Harris S. et. al. (1994), supra). In studies of primary cultures of fetal rat calvarial osteoblasts, BMPs 1, 2, 3, 4, and 6 are expressed by cultured cells prior to the formation of mineralized bone nodules (Harris S. et al. (1994), supra). Like alkaline phosphatase, osteocalcin and osteopontin, the BMPs are expressed by cultured osteoblasts as they proliferate and differentiate.

Although the BMPs are potent stimulators of bone formation in vitro and in vivo, there are disadvantages to their use as therapeutic agents to enhance bone healing. Receptors for the bone morphogenetic proteins have been identified in many tissues, and the BMPs themselves are expressed in a large variety of tissues in specific temporal and spatial patterns. This suggests that BMPs may have effects on many tissues in addition to bone, potentially limiting their usefulness as therapeutic agents when administered systemically. Moreover, since they are peptides, they would have to be administered by injection. These disadvantages impose severe limitations to the development of BMPs as therapeutic agents.

There is a plethora to conditions that are characterized by the need to enhance bone formation. Perhaps the most obvious is the case of bone fractures, where it would be desirable to stimulate bone growth and to hasten and complete bone repair Agents that enhance bone formation would also be useful in facial reconstruction procedures. Other bone deficit conditions include bone segmental detects, periodontal disease, metastatic bone disease, osteolytic bone disease and conditions where connective tissue repair would be beneficial, such as healing or regeneration of cartilage defects or injury. Also of great significance is the chronic condition of osteoporosis, including age-related osteoporosis and osteoporosis associated with post-menopausal hormone status. Other conditions characterized by the need for bone growth include primary and secondary hyperparathyroidism, disuse osteoporosis, diabetes-related osteoporosis, and glucocorticoid-related osteoporosis.

There are currently no satisfactory pharmaceutical approaches to managing any of these conditions. Bone fractures are still treated exclusively using casts, braces, anchoring devices and other strictly mechanical means. Further bone deterioration associated with post-menopausal osteoporosis has been treated with estrogens or bisphosphonates, which have known side effects.

U.S. Pat. No. 5,280,040 discloses compounds described as useful in the treatment of osteoporosis. These compounds putatively achieve this result by preventing bone resorption.

Wang, G. -J. et al, J Formos Med Assoc (1995) 94:589-592 report that certain lipid clearing agents, exemplified by lovastatin and bezafibrate, were able to inhibit the bone resorption resulting from steroid administration in rabbits. There was no effect on bone formation by these two compounds in the absence of steroid treatment. The mechanism of the inhibition in bone resorption observed in the presence of steroids (and the mechanism of the effect of steroid on bone per se) is said to be unknown. The authors state that steroid-induced bone loss is associated with a decrease in bone formation attributed to an inhibitory effect of corticosteroid on osteoblast activity and an increase in bone absorption due to direct osteoclast stimulation and to an indirect inhibition of intestinal calcium absorption with a secondary increase in parathyroid hormone production. Other mechanisms mentioned include those attributable to lipid abnormalities and hyperlipidemia which lead to circulatory impairment, obstruction of subchonidral vessels, osteocyte necrosis and osteoporosis. In light to the known activities of lovastatin and bezafibrate, the authors attribute the effect on bone loss to their ability to lower lipid levels and overcome the impairment to circulation within the femoral head. There is no suggestion in Wang et al. that lovastatin directly enhances bone formation.

An abstract entitled “Lovastatin Prevents Steroid-Induced Adipogenesis and Osteoporosis” by Cui, Q. et al. appeared in the Reports of the ASBMR 18th Annual Meeting (September 1996) J. Bone Mineral Res. (1996) 11(Sl):S510. The abstract reports that lovastatin diminished triglyceride vesicles that accumulated when osteoprogenitor cells cloned from bone marrow stroma of chickens were treated in culture with dexamethasone. Lovastatin was reported to diminish the expression of certain mRNAs and to allow the cells to maintain the osteogenic phenotype after dexamethasone treatment. Further, chickens that had undergone bone loss in the femoral head as a result of dexamethasone treatment were improved by treatment with lovastatin. Again, there is no suggestion that lovastatin directly enhances bone formation in the absence of steroid treatment.

In any event, these data are contrary to reports that dexamethasone and other inducers, such as BMPs, induce osteoblastic differentiation and stimulate osteocalcin MRNA (Bellows, C. G., et al., Develop Biol (1990) 140:132-38; Rickard, D. J., et al., Develop Biol (1994) 161:218-28). In addition, Ducy, P. et al., Nature (1996) 382:448-52 have recently reported that osteocalcin deficient mice exhibit a phenotype marked by increased bone formation and bones of improved functional quality, without impairment of bone resorption. Ducy et al. state that their data suggest that osteocalcin antagonists may be of therapeutic use in conjunction with estrogen replacement therapy (for prevention or treatment of osteoporosis).

Other references which may suggest the use of various compounds for such treatments include the following: U.S. Pat. No. 5,773,428; WO97/15561; WO98/0884(); WO96/24350 and DE 4338944.

The oxazolidinones are a new class of antibacterial agents useful in treating gram positive bacterial infections. Representative of this class is the compound known as linezolid, developed by Pharmacia & Upjohn. Oxazolidinones having a thiocarbonyl functionality have been described in WO98/54161 and PCT/US98/25308. However, the use of these compounds for the treatment of osteoporosis, bone resorption or other bone diseases has not been disclosed nor suggested.

Despite the above teachings, there still exists a need in the art for compounds useful for stimulating bone formation without the drawbacks associated with presently known treatments for bone deficit conditions.

### BRIEF SUMMARY OF THE INVENTION

In accordance with the present invention a novel method for treating osteoporosis, bone resorption or other bone diseases without the drawbacks associated with presently known treatments for bone deficit conditions is provided. More specifically, the inventive method comprises the administration of oxazolidinones having a thiocarbonyl functionality to the patient in need thereof.

In one embodiment, the present invention provides a method for treating or preventing osteoporosis, bone resorption or any other bone disease in a vertebrate mammal comprising the step of administering to the mammal in need of such treatment an effective amount of a compound of formula I

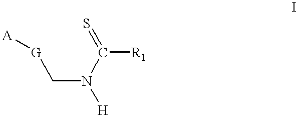

or pharmaceutical acceptable salts thereof wherein:

G is

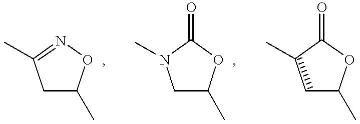

R₁ is

a) H,

b) NH₂,

c) NH—C₁, alkyl,

d) C₁₋₄ alkyl,

e) —OC₁₋₄ alkyl,

f) —S C₁₋₄ alkyl,

g) C₁₋₄ alkyl substituted with 1-3 F, 1-2 Cl, CN or —COOC₁₋₄ alkyl,

h) C₃₋₆ cycloallyl,

i) N(C₁₋₄ alkyl)₂ or

j) N(CH₂)₂₋₅;

A is

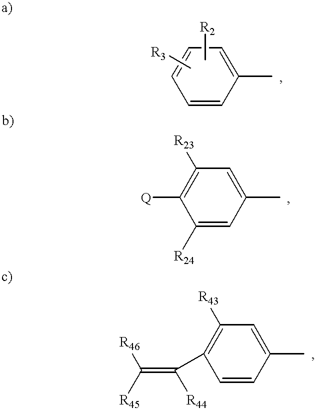

d) a 5-membered heteroaromatic moiety having one to three atoms selected from the group consisting of S, N, and O,

wherein the 6-membered heteroaromatic moiety is bonded via a carbon atom,

wherein the 6-membered heteroaromatic moiety can additionally have a fused-on benzene or naphthyl ring,

wherein the heteroaromatic moiety is optionally substituted with one to three R₄₈,

e) a 6-membered heteroaromatic moiety having at least one nitrogen atom,

wherein the heteroaromatic moiety is bonded via a carbon A. atom,

wherein the 6-membered heteroaromatic moiety can additionally have a fused-on benzene or naphthyl ring,

wherein the heteroaromatic moiety is optionally substituted with one to three R₅₅,

f) a β-carbolin-3-yl, or indolizinyl bonded via the 6-membered ring, optionally substituted with one to three R₅₅,

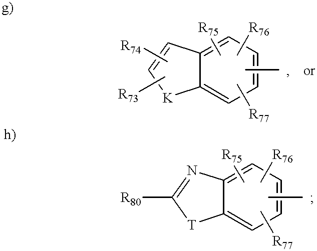

wherein R₂ is

a) H,

b) F,

c) Cl,

d) Br

e) C₁₋₃ alkyl,

f) NO₂, or

g) R₂ and R₃ taken together are —O—(CH₂)ₙ—O—;

R₃ is

a) —S(═O)ᵢ R₄,

b) —S(═O)₂—N═S(O)ⱼR₅R₆,

c) —SC(═O)R₇,

d) —C(═O)R₈,

e) —C(═O)R₉,

f) —C(═O)NR₁₀R₁₁,

g) —C(═NR₁₂)R₈,

h) —C(R₈)(R₁₁)—OR₁₃,

i) —C(R₉)(R₁₁)—OR₁₃,

j) —C(R₈)(R₁₁)—OC(═O)R₁₃,

k) —C(R₉)(R₁₁)—OC(═O)R₁₃,

l) —NR₁₀R₁₁,

m) —N(R₁₀)—C(═O)R₇,

n) —N(R₁₀)—S(═O)ᵢR₇,

o) —C(OR₁₄)(OR₁₅)R₈,

p) —C(R₈)(R₁₆)—NR₁₀R₁₁, or

q) C₁₋₈ alkyl substituted with one or more ═O other than at alpha position, —S(═O)ᵢR₁₇, —NR₁₀R₁₁, C₂₋₅ alkenyl, or C₂₋₅ alkynyl;

R₄ is

a) C₁₋₄ alkyl optionally substituted with one or more halos, OH, CN, NR₁₀R₁₁, or —CO₂R₁₃,

b) C₂₋₄ alkenyl,

c) —NR₁₆R₁₈,

d) —N₃,

e) —NHC(═O)R₇,

f) —NR₂₀C(═O)R₇,

g) —N(R₁₉)₂,

h) —NR₁₆R₁₉, or

i) —NR₁₉R₂₀,

R₅ and R₆ at each occurrence are the same or different and are

a) C₁₋₂ alkyl, or

b) R₅ and R₆ taken together are —(CH₂)ₖ—;

R₇ is C₁₋₄ alkyl optionally substituted with one or more halos;

R₈ is

a) H, or

b) C₁₋₄ alkyl optionally substituted with one or more halos, or C₃₋₈ cycloalkyl;

R₉ is C₁₋₄ alkyl substituted with one or more

a) —S(═O)R₁₇,

b) —OR₁₃,

c) —OC(═O)R₁₃,

d) —NR₁₀R₁₁, or

e) C₁₋₅ alkenyl optionally substituted with CHO;

R₁₀ and R₁₁ at each occurrence are the same or different and are

a) H,

b) C₁₋₄ alkyl, or

c) C₃₋₈ cycloalkyl;

R₁₂ is

a) —NR₁₀R₁₁,

b) —OR₁₀; or

c) —NHC(═O)R₁₀;

R₁₃ is

a) H, or

b) C₁₋₄ alkyl;

R₁₄ and R₁₅ at each occurrence are the same or different and are

a) C₁₋₄ alkyl, or

b) R₁₄ and R₁₅ taken together are —(CH)ₗ—;

R₁₆ is

a) H,

b) C₁₋₄ alkyl, or

c) C₃₋₈ cycloalkyl;

R₁₇ is

a) C₁₋₄ alkyl, or

b) C₃₋₈ cycloalkyl;

R₁₈ is

a) H,

b) C₁₋₄ alkyl,

c) C₂₋₄ alkenyl,

d) C₃₋₄ cycloalkyl,

e) —OR₁₃ or

f) —NR₂₁R₂₂;

R₁₉ is

a) Cl,

b) Br, or

c) I;

R₂₀ is a physiologically acceptable cation;

R₂₁ and R₂₂ at each occurrence are the same or different and are

a) H,

b) C₁₋₄ alkyl, or

c) —NR₂₁R₂₂ taken together are —(CH₂)ₘ—;

wherein R₂₃ and R₂₄ at each occurrence are the same or different and are

a) H,

b) F,

c) Cl,

d) C₁₋₂ alkyl,

e) CN

f) OH,

g) C₁₋₂ alkoxy,

h) nitro, or

i) amino;

Q is

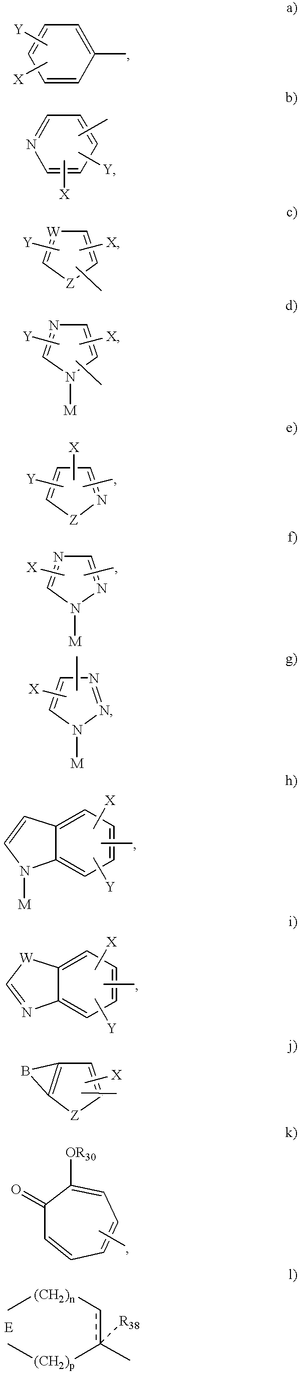

m) a diazinyl group optionally substituted with X and Y,

n) a triazinyl group optionally substituted with X and Y,

o) a quinolinyl group optionally substituted with X and Y,

p) a quinoxalinyl group optionally substituted with X and Y,

q) a naphthyridinyl group optionally substituted with X and Y,

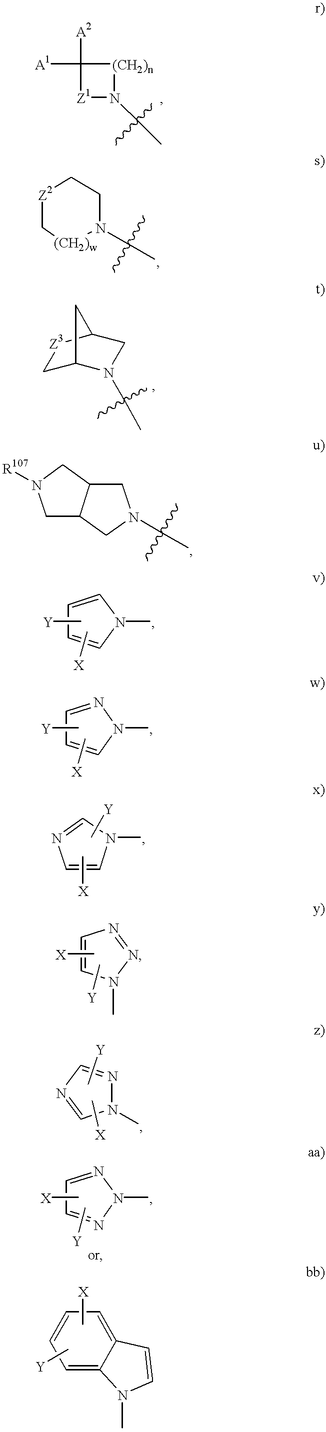

Q and R₂₄ taken together are

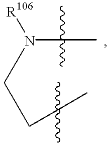

wherein Z¹ is

a) —CH₂—,

b) —CH(R¹⁰⁴)—CH₂—,

c) —C(O)—, or

d) —CH₂CH₂CH₂—;

wherein Z² is

a) —O₂S—,

b) —O—,

c) —N(R¹⁰⁷)—,

d) —OS—, or

e) —S—;

wherein Z³ is

a) —O₂S—,

b) —O—,

c) —OS—, or

d) —S—;

wherein A¹ is

a) H—, or

b) CH₃;

wherein A² is

a) H—,

b) HO—,

c) CH₃—,

d) CH₃O—,

e) R¹⁰²O—CH₂—C(O)—NH—

f) R¹⁰³O—C(O)—NH—,

g) (C₁-C₂)alkyl—O—C(O)—,

h) HO—CH₂—,

i) CH₃O—NH—,

j) (C₁-C₃)alkyl-O₂C—

k) CH₃—C(O)—,

l) CH₃—C(O)—CH₂—,

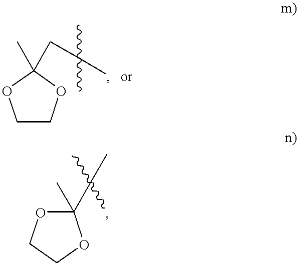

A¹ and A² taken together are:

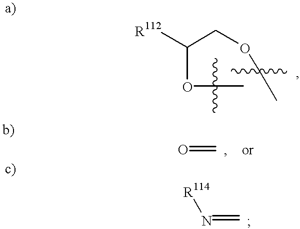

wherein R¹² is

a) H,

b) CH₃—,

C) phenyl-C₂—, or

wherein R¹⁰³ is

a) (C₁-C₃)alkyl-, or

b) phenyl-;

wherein R¹⁰⁴ is

a) H—, or

b) HO—;

wherein R¹⁰⁵ is

a) H—,

b) (C₁-C₃)alkyl-,

c) CH₂═CH—CH₂—, or

d) CH₃—O—(CH₂)₂—;

wherein R¹⁰⁶ is

a) CH₃C(O)—,

b) H—C(O)—,

c) Cl₂CH—C(O)—,

d) HOCH₂—C(O)—,

e) CH₃SO₂—,

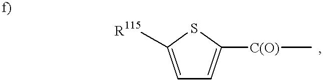

g) F₂CHC(O)—,

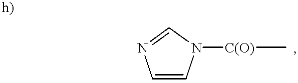

i) H₃C—C(O)—O—CH₂—C(O)—,

j) H—C(O)—O—CH₂—C(O)—,

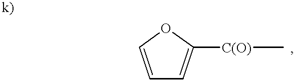

l) HC═C—CH₂O—CH₂—C(O)—, or

m) phenyl-CH₂—O—CH₂—C(O)—;

wherein R¹⁰⁷ is

a) R¹⁰²O—C(R¹¹⁰)(R¹¹¹)—C(O)—,

b) R¹⁰³O—C(O)—,

c) R¹⁰⁸—C(O)—,

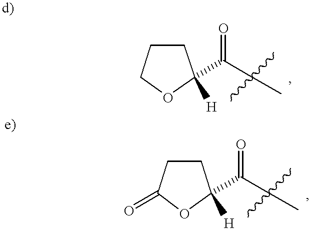

f) H₃C—C(O)—(CH₂)₂—C(O)—,

g) R¹⁰⁹—SO₂—,

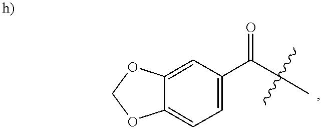

i) HO—CH₂—C(O)—,

i) R¹¹⁶—(CH₂)₂—,

k) R¹¹³—C(O)—O—CH₂—C(O)—,

l) (CH₃)₂N—CH₂—C(O)—NH—,

m) NC—CH₂—,

n) F₂—CH—CH₂—, or

o) R¹⁶⁰R¹⁶¹NSO₂

wherein R¹⁰⁸ is

a) H—,

b) (C₁-C₄)alkyl,

c) aryl —(CH₂)ₚ,

d) ClH₂C—,

e) Cl₂HC—,

f) FH₂C—,

g) F₂HC—,

h) (C₃-C₆)cycloalkyl, or

i) CNCH2—.

wherein R¹⁰⁹ is

a) alkylC₁-C₄,

b) —CH₂Cl

c) —CH₂CH═CH₂,

d) aryl, or

e) —CH₂CN;

wherein R¹¹⁰ and R¹¹¹ are independently

a) H—,

b) CH₃—; or

wherein R¹¹² is

a) H—,

b) CH₃O—CH₂O—CH₂—, or

c) HOCH₂—;

wherein R¹¹³ is

a) CH₃—,

b) HOCH₂—,

c) (CH₃)₂N-phenyl, or

d) (CH₃)₂N—CH₂—;

wherein R¹¹⁴ is

a) HO—,

b) CH₃O—,

c) H₂N—,

d) CH₃O—C(O)—O—,

e) CH₃—C(O)—O—CH₂—C(O)—O—,

f) phenyl—CH₂—O—CH₂—C(O)—O—,

g) HO—(CH₂)₂—O—,

h) CH₃O—CH₂—O—(CH₂)₂—O—, or

i) CH₃O—CH₂—O—; wherein R¹¹³ is

a) CH₃—,

b) HOCH₂—,

c) (CH₃)₂N-phenyl, or

d) (CH₃)₂N—CH₂—;

wherein R¹¹⁵ is

a) H—, or

b) Cl—;

wherein R¹¹⁶ is

a) HO—

b) CH₃O—, or

c) F;

wherein R¹⁵⁰ and R¹⁵¹ are each H or alkyl C₁-C₄ or R¹⁵⁰ and R¹⁵¹ taken together with the nitrogen atom to which each is attached form a monocyclic heterocyclic ring having from 3 to 6 carbon atoms;

B is an unsaturated 4-atom linker having one nitrogen and three carbons;

M is

a) H,

b) C₁₋₈ alkyl,

c) C₃₋₈ cycloallyl,

d) —(CH₂)ₘOR₁₃, or

e) —(CH₂)ₕ—NR₂₁R₂₂;

Z is

a) O,

b) S, or

c) NM;

W is

a) CH,

b) N, or

c) S or O when Z is NM;

Y is

a) H,

b) F,

c) Cl,

d) Br,

e) C₁₋₃ alkyl, or

f) NO₂;

X is

a) H,

b) —CN,

c) OR₂₇,

d) halo,

e) NO₂,

f) tetrazoyl,

g) —SH,

h) —S(═O)ᵢR₄,

i) —S(═O)₂—N═S(O)ⱼR₅R₆,

j) —SC(═O)R₇,

k) —C(═O)R₂₅,

l)—C(═O)NR₂₇R₂₈,

m) —C(═NR₂₉)R₂₅,

n) —C(R₂₅)(R₂₈)—OR₁₃,

o) —C(R₂₅)(R₂₈)—OC(═O)R₁₃,

p)—C(R₂₆)(OR₁₃)—(CH₂)ₕ—NR₂₇R₂₈,

q) —NR₂₇R₂₈,

r) —N(R₂₇)C(═O)R₇,

s) —N(R₂₇)—S(═O)ᵢR₇,

t) —C(OR₁₄)(OR₁₅)ZR₂₈,

u) —C(R₂₅)(R₁₆)—NR₂₇R₂₆, or

v) C₁₋₈ alkyl substituted with one or more halos, OH, ═O other than at alpha position, —S(═O)ᵢR₁₇, —NR₂₇R₂₈, C₂₋₅ alkenyl, C₂₋₅ alkynyl, or C₃₋₈ cycloalkyl;

R₄, R₅, R₆, R₇, R₁₃, R₁₄, R₁₅, R₁₆, and R₁₇ are the same as defined above;

R₂₅ is

a) H,

b) C₁₋₈ alkyl optionally substituted with one or more halos, C₃₋₈ cycloalkyl, C₁₋₄ alkyl substituted with one or more of —S(═O)ᵢR₁₇, —OR₁₈, or OC(═O)R₁₃, NR₂₇R₂₈, or

c) C₂₋₅ alkenyl optionally substituted with CHO, or CO₂R₁₃;

R₂₆ is

a) R₂₈, or

b) NR₂₇N₂₈;

R₂₇ and R₂₈ at each occurrence are the same or different and are

a) H,

b) C₁₋₄ alkyl,

c) C₃₋₈ cycloalkyl,

d) —(CH₂)ₘOR₁₃,

e) —(CH₂)ₕ—NR₂₁R₂₂, or

f) R₂₇ and R₂₈ taken together are —(CH₂)₂O(CH₂)₂—, —(CH₂)ₕCH(COR₇)—, or —(CH₂)₂N(CH₂)₂(R₇);

R₂₉ is

a) —NR₂₇R₂₈,

b) —OR27, or

c) —NHC(═O)R₂₈;

wherein R₃₀ is

a) H,

b) C₁₋₈ alkyl optionally substituted with one or more halos, or

c) C₁₋₈ alkyl optionally substituted with one or more OH, or C₁₋₆ alkoxy,

wherein E is

a) NR₃₉,

b) —S(═O)ᵢ, or

c) O;

R₃₈ is

a) H,

b) C₁₋₆ alkyl,

c) —(CH₂)_{q}-aryl, or

d) halo;

R₃₉ is

a) H,

b) C₁₋₆ alkyl optionally substituted with one or more OH, halo, or —CN,

c) —(CH₂)_{q}-aryl,

d) —CO₂R₄₀,

e) —COR₄₁,

f) —C(═O)—(CH₂)_{q}—C(═O)R₄₀,

g) —S(═O)₂—C₁₋₆ alkyl,

h) —S(═O)₂—(CH₂)_{q}-aryl, or

i) —(C═O)ⱼ-Het;

R₄₀ is

a) H,

b) C₁₋₆ alkyl optionally substituted with one or more OH, halo, or —CN,

c) —(CH₂)_{q}-aryl, or

d) —(CH₂)_{q}—OR₄₂;

R₄₁ is

a) C₁₋₆ alkyl optionally substituted with one or more OH, halo, or —CN,

b) —(CH₂)_{q}-aryl, or

c) —(CH₂)_{q}—OR₄₂;

R₄₂ is

a) H,

b) C₁₋₆ alkyl,

c) —(CH₂)_{q}-aryl, or

d) —C(═O)—C₁₋₆ alkyl;

aryl is

a) phenyl,

b) pyridyl, or

c) napthyl; a to c optionally substituted with one or more halo, —CN, OH, SH, C₁₋₆ alkyl, C₁₋₆ alkoxy, or C₁₋₆ alkylthio;

wherein R43 is

a) H,

b) C₁₋₂ akyl,

c) F, or

d) OH;

R₄₄ is

a) H,

b) CF₃,

c) C₁₋₃ alkyl optionally substituted with one or more halo,

d) phenyl optionally substituted with one or more halo,

e) R₄₄ and R₄₅ taken together are a 5-, 6-, or 7-membered ring of the formula,

or

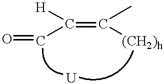

f) R₄₄ and R₄₅ taken together are —(CH₂)ₖ—, when R₄₆ is an electron-withdrawing group;

R₄₅ and R₄₆ at each occurrence are the same or different and are

a) an electron-withdrawing group,

b) H,

c) CF₃,

d) C₁₋₃ alkyl optionally substituted with one halo,

e) phenyl, provided at least one of R₄₅ or R₄₆ is an electron-withdrawing group, or

f) R_{45 and R}₄₆ taken together are a 5-, 6-, 7-membered ring of the formula

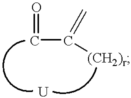

U is

a) CH₂,

b) O,

C) S, or

d) NR₄₇;

R₄₇ is

a) H, or

b) C₁₋₅ alkyl;

wherein R₄₈ is

a) carboxyl,

b) halo,

c) —CN,

d) mercapto,

e) formyl,

f) CF₃,

g) —NO₂,

h) C₁₋₆ alkoxy,

i) C₁₋₆ alkoxyearbonyl,

j) C₁₋₆ alkythio,

k) C₁₋₆ acyl,

l) —NR₄₉ R₅₀,

m) C₁₋₆ alkyl optionally substituted with OH, C₁₋₅ alkoxy, C₁₋₅ acyl, or —NR₄₉R₅₀,

n) C₂₋₈ alkenylphenyl optionally substituted with one or two R₅₁,

o) phenyl optionally substituted with one or two R₅₁,

p) a 5-, or 6-membered (un)saturated heterocyclic moiety having one to three atoms selected from the group consisting of S, N, and O, optionally substituted with one or two R₅₁, or

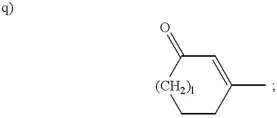

R₄₉ and R₅₀ at each occurrence are the same or different and are

a) H,

b) C₁₋₄ alkyl,

c) C₅₋₆ cycloalkyl, or

d) R₄₉ and R₅₀ taken together with the nitrogen atom is a 5-, 6-membered saturated heterocyclic moiety which optionally has a further hetero atom selected from the group consisting of S, N, and O, and can in turn be optionally substituted with, including on the further nitrogen atom, C₁₋₃ alkyl, or C₁₋₃ acyl;

R₅₁ is

a) carboxyl,

b) halo,

c) —CN,

d) mercapto,

e) formyl,

f) CF₃,

g) —NO₂,

h) C₁₋₆ alkoxy,

i) C₁₋₆ alkoxycarbonyl,

j) C₁₋₆ alkythio,

k) C₁₋₆ acyl,

l) C₁₋₅ alkyl optionally substituted with OH, C₁₋₅ alkoxy, C₁₋₅ acyl, or —NR₄₉R₅₀,

m) phenyl,

n) —C(═O)NR₅₂R₅₃,

o) —NR₄₉R₅₀,

p) —N(R₅₂)(—SO₂R₅₄),

q) —SO₂—NR₅₂R₅₃, or

r) —S(═O)ᵢR₅₄;

R₅₂ and R₅₃ at each occurrence are the same or different and are

a) H,

b) C₁₋₅ alkyl, or

c) phenyl;

R₅₄ is

a) C₁₋₄ alkyl, or

b) phenyl optionally substituted with C₁₋₄ alkyl;

wherein R₅₅ is

a) carboxyl,

b) halo,

c) —CN,

d) mercapto,

e) formyl,

f) CF₃,

g) —NO₂,

h) C₁₋₆ alkoxy,

i) C₁₋₆ alkoxyearbonyl,

j) C₁₋₆ alkythio

k) C₁₋₆ acyl,

l) —NR₅₆R₅₇,

m) C₁₋₆ alkyl optionally substituted with OH, C₁₋₅ alkoxy, C₁₋₅ acyl, or —NR₅₆R₅₇,

n) C₂₋₈ alkenylphenyl optionally substituted with one or two R₅₈,

o) phenyl optionally substituted with one or two R₅₈,

p) a 5- or 6-membered (un)saturated heterocyclic moiety having one to three atoms selected from the group consisting of S, N, and O, optionally substituted with one or two R₅₈, or

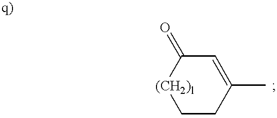

R₅₆ and R₅₇ at each occurrence are the same or different and are

a) H,

b) formyl,

c) C₁₋₄ alkyl,

d) C₁₋₄ acyl,

e) phenyl,

f) C₃₋₆ cycloalkyl, or

g) R₅₆ and R₅₇ taken together with the nitrogen atom is a 5-, 6-membered saturated heterocyclic moiety which optionally has a further hetero atom selected from the group consisting of S, N, and O, and can in turn be optionally substituted with, including on the further nitrogen atom, phenyl, pyramidal, C₁₋₃ alkyl, or C₁₋₃ acyl;

R₅₈ is

a) carboxyl,

b) halo,

c) —CN,

d) mercapto,

e) formyl,

f) CF₃,

g) —NO₂,

h) C₁₋₆ alkoxy,

i) C₁₋₆ alkoxycarbonyl,

j) C₁₋₆ alkythio,

k) C₁₋₆ acyl,

l) phenyl,

m) C₁₋₆ alkyl optionally substituted with OH, azido, C₁₋₅ alkoxy, C₁₋₅ acyl, —NR₆₅R₆₆, —SR₆₇, —O—SO₂R₆₈, or

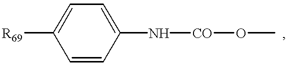

n) —C(═O)NR₅₉R₆₀,

o) —NR₅₆R₅₇,

p) —N(R₅₉)(—SO₂R₅₄),

q) —SO₂—NR₅₉R₆₀,

r) —S(═O)ᵢR₅₄,

s) —CH═N—R₆₁, or

t) —CH(OH)—SO₃R₆₄;

R₆₄ is the same as defined above;

R₅₉ and R₆₀ at each occurrence are the same or different and are

a) H,

b) C₁₋₆ alkyl,

c) phenyl, or

d) tolyl;

R₆₁ is

a) OH,

b) benzyloxy,

c) —NH—C(═O)—NH₂,

d) —NH—C(═S)—NH₂, or

e) —NH—C(═NH)—NR₆₂R₆₃;

R₆₂ and R₆₃ at each occurrence are the same or different and are

a) H, or

b) C₁₋₄ alkyl optionally substituted with phenyl or pyridyl;

R₆₄ is

a) H, or

b) a sodium ion;

R₆₅ and R₆₆ at each occurrence are the same or different and are

a) H,

b) formyl,

c) C₁₋₄ alkyl,

d) C₁₋₄ acyl,

e) phenyl,

f) C₃₋₆ cycloalkyl,

g) R₆₅ and R₆₆ taken together are a 5-, 6-membered saturated heterocyclic moiety having one to three atoms selected from the group consisting of S, N, and O, optionally substituted with, including on the nitrogen atom, phenyl, pyrimidyl, C₁₋₃ alkyl, or C₁₋₃ acyl,

h) —P(O)(OR₇₀)(OR₇₁), or

i) —SO₂—R₇₂;

R₆₇ is

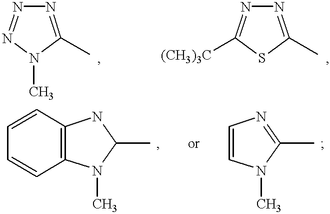

R₆₈ is C₁₋₃ alkyl;

a) C₁₋₆ alkoxycarbonyl, or

b) carboxyl;

R₇₀ and R₇₁ at each occurrence are the same or different and are

a) H, or

b) C₁₋₃ alkyl;

R₇₂ is

a) methyl,

b) phenyl , or

c) tolyl;

wherein K is

a) O, or

b) S;

R₇₃, R₇₄, R₇₅, R₇₆, and R₇₇ at each occurrence are the same or different and are

a) H,

b) carboxyl,

c) halo,

d) —CN,

e) mereapto,

f) formyl,

g) CF₃,

h) —NO₂,

i) C₁₋₆ alkoxy,

j) C₁₋₆ alkoxycarbonyl,

k) C₁₋₆ alkythio,

l) C₁₋₆ acyl,

m) —NR₇₈R₇₉,

n) C₁₋₆ alkyl optionally substituted with OH, C₁₋₅ alkoxy, C₁₋₅ acyl, —NR₇₈R₇₉, —N(phenyl)(CH₂—CH₂—OH), —O—CH(CH₃)(OCH₂CH₃), or —O-phenyl-[para-NHC(═O)CH₃],

o) C₂₋₈ alkenylphenyl optionally substituted with R₅₁,

p) phenyl optionally substituted with R₅₁, or

q) a 5-, or 6-membered (un)saturated heterocyclic moiety having one to three atoms selected from the group consisting of S, N, and O, optionally substituted with R₅₁;

R₅₁ is the same as defined above;

R₇₈ and R₇₉ at each occurrence are the same or different and are

a) H,

b) C₁₋₄ alkyl,

c) phenyl, or

d) R₇₈ and R₇₉ taken together with the nitrogen atom is a 5-, 6-membered saturated heterocyclic moiety which optionally has a further hetero atom selected from the group consisting of S, N, and O, and can in turn be optionally substituted with, including on the further nitrogen atom, C₁₋₃ alkyl, or C₁₋₃ acyl;

wherein T is

a) O,

b) S, or

c) SO₂;

R₇₅, R₇₆, and R₇₇ are the same as defined above;

R₈₀ is

a) H,

b) formyl,

c) carboxyl,

d) C₁₋₆ alkoxycarbonyl,

e) C₁₋₈ alkyl,

f) C₁₋₈ alkenyl, wherein the substitutes (e) and (f) can be optionally substituted with OH, halo, C₁₋₆ alkoxy, C₁₋₆ acyl, C₁₋₆ alkylthio or C₁₋₆ alkoxyearbonyl, or phenyl optionally substituted with halo,

g) an aromatic moiety having 6 to 10 carbon atoms optionally substituted with carboxyl, halo, —CN, formyl, CF₃, —NO₂, C₁₋₆ alkyl, C₁₋₆ alkoxy, C₁₋₆ acyl, C₁₋₆ alkylthio, or C₁₋₆ alkoxycarbonyl;

h) —NR₈₁R₈₂,

i) —OR₉₀,

j) —S(═O)ᵢ—R₉₁,

k) —SO₂—N(R₉₂)(R₉₃), or

l) a radical of the following formulas:

R₈₁ and R₈₂ at each occurrence are the same or different and are

a) H,

b) C₃₋₆ cycloalkyl,

c) phenyl,

d) C₁₋₆ acyl,

e) C₁₋₈ alkyl optionally substituted with OH, C₁₋₆ alkoxy which can be substituted with OH, a 5-, or 6-membered aromatic heterocyclic moiety having one to three atoms selected from the group consisting of S, N, and O, phenyl optionally substituted with OH, CF₃, halo, —NO₂, C₁₋₄ alkoxy, —NR₈₃R₈₄, or

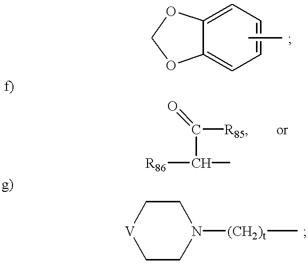

V is

a) O,

b) CH₂, or

C) NR₈₇;

R₈₃ and R₈₄ at each occurrence are the same or different and are

a) H, or

b) C₁₋₄ alkyl;

R₈₅ is

a) OH,

b) C₁₋₄ alkoxy, or

c) —NR₈₈R₈₉;

R₈₆ is

a) H, or

b) C₁₋₇ alkyl optionally substituted with indolyl, OH, mercaptyl, imidazoly, methylthio, amino, phenyl optionally substituted with OH, —C(═O)—NH₂, —CO₂H, or —C(═NH)—NH₂;

R₈₇ is

a) H,

b) phenyl, or

c) C₁₋₆ alkyl optionally substituted by OH;

R₈₈ and R₈₉ at each occurrence are the same or different and are

a) H,

b) C₁₋₅ alkyl

c) C₃₋₆ cycloalky, or

d) phenyl;

R₉₀ is

a) C₁₋₅ alkyl optionally substituted with C₁₋₆ alkoxy or C₁₋₆ hydroxay, C₃₋₆ cycloalkyl, a 6-membered aromatic optionally benzo-fused heterocyclic moiety having one to three nitrogen atoms, which can in turn be substituted with one or two —NO₂, CF₃, halo, —CN, OH, C₁₋₅ alkyl, C₁₋₅ alkoxy, or C₁₋₄ acyl;

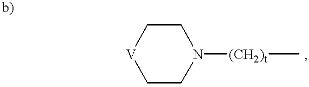

c) phenyl, or

d) pyridyl;

R₉₁ is

a) C₁₋₁₆ alkyl,

b) C₁₋₁₆ alkenyl,

wherein the substitutes (a) and (b) can be optionally substituted with C₁₋₆ alkoxycarbonyl, or a 5-, 6-, 7-membered aromatic heterocyclic moiety having one to three atoms selected from the group consisting of S, N, and O,

c) an aromatic moiety having 6 to 10 carbon atoms, or

d) a 5-, 6-, 7-membered aromatic heterocyclic moiety having one to three atoms selected from the group consisting of S, N, and O,

wherein the substitutes (c) and (d) can be optionally substituted with carboxyl, halo, —CN, formyl, CF₃, —NO₂, C₁₋₆ alkyl, C₁₋₆ alkoxy, C₁₋₆ acyl, C₁₋₆ alkylthio, or C₁₋₆ alkoxycarbonyl;

R₉₂ and R₉₃ at each occurrence are the same or different and are

a) H,

b) phenyl,

c) C₁₋₆ alkyl, or

d) benzyl;

R₉₄ and R₉₅ at each occurrence are the same or different and are

a) H,

b) OH,

c) C₁₋₆ alkyl optionally substituted with —NR₈₃R₈₄, or

d) R₉₄ and R₉₅ taken together are ═O;

R₉₆ is

a) an aromatic moiety having 6 to 10 carbon atoms,

b) a 5-, or 6-membered aromatic optionally benzo-fused heterocyclic moiety having one to three atoms selected from the group consisting of S, N, and O,

wherein the substituents (a) and (b) which can in turn be substituted with one or three —NO₂, CF₃, halo, —CN, OH, phenyl, C₁₋₅ alkyl, C₁₋₅ alkoxy, or C₁₋₅ acyl,

c) morpholinyl,

d) OH,

e) C₁₋₆ alkoxy,

f) —NR₈₃R₈₄,

g) —C(═O)—R₉₇, or

h)

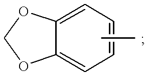

R₉₇ is

a) morpholinyl,

b) OH, or

c) C₁₋₆ alkoxy;

h is 1, 2, or 3;

i is 0, 1, or 2;

j is 0 or 1;

k is 3, 4, or 5;

l is 2 or 3;

m is 4 or 5;

n is 0, 1, 2, 3, 4, or 5;

p is 0, 1, 2, 3, 4, or 5; with the proviso that n and p together are 1, 2, 3, 4, or 5;

q is 1, 2, 3,or4;

r is 2, 3, or 4;

t is 0, 1, 2, 3, 4, 5, or 6;

u is 1 or 2;

w is 0, 1, 2,or 3.

An object of the present invention is to provide a novel method for treating or preventing osteoporosis, bone resorption and other bone diseases.

Still another object of the present invention is to prepare a medicament for treating or preventing osteoporosis, bone resorption and other bone diseases in a mammal

These, and other objects, will readily be apparent to those skilled in the art as reference is made to the detailed description of the preferred embodiment.

### DETAILED DESCRIPTION OF THE PREFERRED EMBODIMENT

In describing the preferred embodiment, certain terminology will be utilized for the sake of clarity. Such terminology is intended to encompass the recited embodiment, as well as all technical equivalents which operate in a similar manner for a similar purpose to achieve a similar result.

The present invention is directed to the use of the compounds of formula I above to treat osteoporosis, bone resorption or other bone diseases. The compounds, including their synthesis, are described in greater detail in WO98/54161 and PCT/US98/25308. To the extent necessary for completion, the disclosure of these documents is expressly incorporated by reference.

The compounds used in the invention can be prepared using known compounds and intermediates of oxazolidinones, isoxazolines and butyolactones as intermediates and synthetic methods known in the art. Thioamides of the invention can typically be prepared by the reaction of the corresponding amide with Lawesson's reagent.

Compounds disclosed in the following publications are suitable intermediates for preparation of the compounds used in this invention and are hereby incorporated by reference for their disclosure of suitable compounds that can be converted to the subject thiocarbonyl derivatives. U.S. Pat. Nos. 5,225,565; 5,182,403; 5,164,510; 5,247,090; 5,231,188; 5,565,571; 5,547,950; and 5,523,403. PCT Application and publications PCT/US93104850, WO94/01110; PCT/US94/08904, WO95/07271; PCT/US95/02972, WO95125106; PCT/US95/10992, WO96/13502; PCT/US96/05202, WO96135691; PCT/US96/12766; PCT/US96/13726; PCT/US96114135; PCT/US96/17120; PCT/US96/19149; PCT/US97/01970; PCT/US95/12751, WO96115130; and PCT/US96/00718, WO96/23788.

Chemical conversion techniques for converting various intermediates having a CH₂NH₂ on the oxazolidinone ring to CH₂NH—C(S)—CH₃ is disclosed by Hartke, K, Barrmeyer, S., J. prakt. Chem. 1996, 338, 251-6. Similarly, conversion of CH₂NHC(═O)CH₃ to CH₂NHC(S)NHCH₃ is reported by Cava, M. P.; Levinson, M. I., Thionation Reactions of Lawesson's Reagents, Tetrahedron 1985, 41, 5061-87.

For the purpose of the present invention, the carbon content of various hydrocarbon containing moieties is indicated by a prefix designating the minimum and maximum number of carbon atoms in the moiety, i.e., the prefix Cᵢ₋ⱼ defines the number of carbon atoms present from the integer “i” to the integer “j”, inclusive. Thus, C₁₋₄ alkyl refers to alkyl of 1-4 carbon atoms, inclusive, or methyl, ethyl, propyl, butyl and isomeric forms thereof.

The terms “C₁₋₂ alkyl”, “C₁₋₃ alkyl”, “C₁₋₄ alkyl”, “C₁₋₅ alkyl”, “C₁₋₆ alkyl”, “C₁₋₈ alkyl”, and “C₁₋₁₆ alkyl” refer to an alkyl group having one to two, one to three, one to four, one to five, one to six, one to eight, or one to sixteen carbon atoms respectively such as, for example, methyl, ethyl, propyl, butyl, pentyl, hexyl, heptyl, octyl, nonyl, decyl, undecyl, dodecyl, tridecyl, tetradecyl and their isomeric forms thereof.

The terms “C₂₋₄ alkenyl”, “C₂₋₅ alkenyl”, “C₂₋₈ alkenyl”, “C₂₋₁₄ alkenyl” and “C₂₋₁₆ alkenyl” refer to at least one double bond alkenyl group having two to four, two to five, two to eight, two to fourteen, or two to sixteen carbon atoms, respectively such as, for example, ethenyl, propenyl, butenyl, pentenyl, pentdienyl, hexenyl, hexdienyl, heptenyl, heptdienyl, octenyl, octdienyl, octatrienyl, nonenyl, nonedienyl, nonatrienyl, undecenyl, undecdienyl, dodecenyl, tridecenyl, tetradecenyl and their isomeric forms thereof.

The terms “C₂₋₅ alkynyl”, “C₂₋₈ alkynyl”, and “C₂₋₁₀ alkynyl” refer to at least one triple bond alkynyl group having two to five, two to eight, or two to ten carbon atoms respectively such as, for example, ethynyl propynyl, butynyl pentynyl, pentdiynyl, hexynyl, hexdiynyl heptynyl, heptdiynyl, octynyl, octdiynyl, octatriynyl, nonynyl, nonediynyl, nonatriynyl and their isomeric forms thereof.

The terms “C₃₋₄ cycloalkyl”, “C₃₋₆ cycloalkyl”, “C₅₋₆ cycloalkyl”, and “C₃₋₈ cycloalkyl” refer to a cycloalkyl having three to four, three to six, five to six, or three to eight carbon atoms respectively such as, for example, cyclopropyl, cyclobutyl, cyclopentyl, cyclohexyl, cycloheptyl, cyclooctyl, and their isomeric forms thereof.

The terms “C₁₋₄ alkoxy”, “C₁₋₆ alkoxy”, and “C₁₋₈ alkoxy” refer to an alkyl group having one to four, one to six, or one to eight carbon atoms respectively attached to an oxygen atom such as, for example, methoxy, ethoxy, propyloxy, butyloxy, pentyloxy, hexyloxy, heptyloxy, or octyloxy and their isomeric forms thereof.

The terms “C₁₋₆ alkylamino”, and “C₁₋₈ alkylamino” refer to an alkyl group having one to six, or one to eight carbon atoms respectively attached to an amino moiety such as, k)r example, methylamino, ethylamino, propylamino, butylamino, pentylamino, hexylamino, heptylamino, or octoylamino and their isomeric forms thereof.

The terms “C₁₋₆ dialkylamino”, and “C₁₋₈ dialkylamino” refer to two alkyl groups having one to six, or one to eight carbon atoms respectively attached to an amino moiety such as, for example, dimethylamino, methylethylamino, diethylamino, dipropylamino, methypropylamino, ethylpropylamino, dibutylarnino, dipentylamino, dihexylamino, methylhecylamino, diheptylamino, or dioctoylamino and their isomeric forms thereof.

The terms “C₁₋₃ acyl”, “C₁₋₄ acyl”, “C₁₋₅ acyl”, “C₁₋₆ acyl”, “C₁₋₈ acyl”, and “C₂₋₈ acyl” refer to a carbonyl group having an alkyl group of one to three, one to four, one to five, one to six, one to eight, or two to eight carbon atoms.

The terms “C₁₋₄ alkoxycarbonyl”, “C₁₋₆ alkoxycarbonyl”, and “C₁₋₈ alkoxycarbonyl” refer to an ester group having an alkyl group of one to four, one to six, or one to eight carbon atoms.

The term “C₁₋₈ alkyl phenyl” refers to an alkyl group having one to eight carbon atoms and isomeric forms thereof which is substituted with at least one phenyl radical.

The term “C₂₋₈ alkenyl phenyl” refers to a at least one double bond alkenyl group having one to eight carbon atoms and isomeric forms thereof which is substituted with at least one phenyl radical.

The term “C₁₋₈ alkyl pyridyl” refers to an alkyl group having one to eight carbon atoms and isomeric forms thereof which is substituted with at least one pyridyl radical.

The term “C₁₋₈ hydroxyl” refers to an alkyl group having one to eight carbon atoms and isomeric forms thereof attached to a hydroxy group.

The term “C₁₋₈ alkylsulfonyl” refers to an alkyl group having one to eight carbon atoms and isomeric lorms thereof attached to a SO₂ moiety.

The term “C₁₋₆ alkylthio” refers to an alkyl group having one to six carbon atoms and isomeric forms thereof attached to a sulfur atom.

The term “Het” refers to 5 to 10 membered saturated, unsaturated or aromatic heterocyclic rings containing one or more oxygen, nitrogen, and sulfur forming such groups as, for example, pyridine, thiophene, furan, pyrazoline, pyrimidine, 2-pyridyl, 3-pyridyl, 4-pyridyl, 2-pyrimidinyl, 4-pyrimidinyl, 5-pyriridinyl, 3-pyridazinyl, 4-pyridazinyl, 3-pyrazinyl, 2-quinolyl, 3-quinolyl, 1-isoquinolyl, 3-isoquinolyl, 4-isoquinolyl, 2-quinazolinyl, 4-quinazolinyl, 2-quinoxalinyl, I-phthalazinyl, 4-oxo-2-imidazolyl, 2-imidazolyl, 4-imidazolyl, 3-isoxazolyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 4,5,-dihydrooxazole, 1,2,3-oxathiole, 1,2,3-oxadiazole, 1,2,4-oxadiazole, 1,2,5-oxadiazole, 1,3,4-oxadiazole, 2-thiazolyl 4-thiazolyl, 5-thiazolyl, 3-isothiazole, 4-isothiazole, 5-isothiazole, 2-indolyl, 3-indolyl, 3-indazolyl, 2-benzoxazolyl, 2-benzothiazolyl, 2-benzimidazolyl, 2-benzofliranyL 3-benzofuranyl, benzoisothiazole, benzisoxazole, 2-furanyl, 3-furanyl 2-thienyl, 3-thienyl, 2-pyrrolyl, 3-pyrrolyl, 3-isopyrrolyl 4-isopyrrolyl, 5-isopyrrolyL 1,2,3,-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 1-pyrrolyl, l-pyrazolyl, 1,2,3-triazol-1-yl, 1,2,4-triazol-1-yl, l-tetrazolyl, 1-indolyl, 1-indazolyl, 2-isoindolyl, 7-oxo-2-isoindolyl,l-purinyl, 3-isothiazolyl, 4-isothiazolyl and 5-isothiazolyl, 1,3,4,-oxadiazole, 4-oxo-2-thiazolinyl, or 5-methyl-1,3,4-thiadiazol-2-yl, thiazoledione, 1,2,3,4-thiatriazole, 1,2,4-dithiazolone. Each of these moieties may be substituted as appropriate.

The term halo refers to fluoro, chloro, bromo, or iodo.

When Q is the structure of

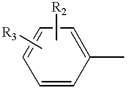

the dotted line in the heterocyclic ring means that this bond can be either single or double. In the case where the dotted line is a double bond, the R₃₉ group will not be present.

The compounds of Formula I of this invention contain a chiral center at C5 of the isoxazoline ring, and as such there exist two enantiomers or a racemic mixture of both. This invention relates to both the enantiomers, as well as mixtures containing both the isomers. In addition, depending on substituents, additional chiral centers and other isomeric forms may be present in any of A or R₁ group, and this invention embraces all possible stereoisomers and geometric forms in these groups.

In even more preferred embodiments, the compounds selected for use are of formula (II):

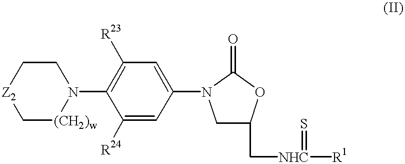

wherein Z₂ is —)₂S—, —O—, —N(R¹⁰⁷)—, —OS—, or —S—;

w iso, 1, 2, or 3;

R²³ and R²⁴ are the same or different and can be H or F; and

R¹ is H, NH₂, NHalkylC₁-C₄; N(alkylC₁-C₄)₂;

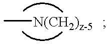

alkylC₁-C₄; OalkylC₁-C₄; SalkylC₁-C₄; alkylC₁-C₄ substituted with l-3F, 1-2Cl,

CN, or —COOalykylC₁-C₄, or cycloalkylC₃-C₆, wherein in each occurrence of the alkyl group may be straight or branched; and

R¹⁰⁷ is

a) R¹⁰²O—C(R¹¹⁰)(R¹¹¹)—C(O)—,

b) R¹⁰³O—C(O)—,

c) R¹⁰⁸—C(O)—,

d) R¹⁰⁹—SO₂—,

e) NC—CH₂—,

f) FCHCH₂—, or

g) R¹⁵⁰R¹⁵¹NSO₂.

wherein R¹⁰² is H, CH₃—, phenyl-CH₂—, or CH₃C(O); each of R¹¹⁰ and R¹¹¹ is selected from H or CH₃; R¹⁰³ is alkylC₁-C₃ or phenyl; R¹⁰⁸ is H, alkylC₁-C₄, aryl(CH₂)₀₋₅, CNCH₂—, ClCH₂—,

Cl₂HC—, FH₂C—, F₂HC—, or cycloalkylC₃-C₆; R¹⁰⁵ and R¹⁵¹ are the same or different and are selected from H, alkylC₁-C₄, or R¹⁵⁰ and R¹⁵¹ taken together with the nitrogen to which each is attached forms a monocyclic heterocyclic ring having from 3 to 6 carbon atoms.

Specifically preferred compounds for use include:

(S)-trans-[[3-[3-Fluoro-4-(tetrahydro-1-oxido-2H-thiopyran-4-yl)phenyl]-2-oxo-5-oxazolidinyl]methyl]thiourea; and

(S)-N-[[3-[3-Fuoro-4(4-thiomorpholinyl)phenyl]-2-oxo-5-oxazolidinyl]methyl]thio-acetamide, thiomorpholine S-oxide.

The compounds used in the present invention can be converted to their salts, where appropriate, according to conventional methods.

The term “pharmaceutically acceptable salts” refers to acid addition salts useful for administering the compounds used in this invention and include hydrochloride, hydrobromide, hydroiodide, sulfate, phosphate, acetate, propionate, lactate, mesylate, maleate, malate, succinate, tartrate, citric acid, 2-hydroxyethyl sulfonate, fumarate and the like. These salts may be in hydrated form.

The compounds are useful for treatment of osteoporosis, bone resorption and other bone diseases in mammals. The term “mammals” is intended to include both humans and non-human vertebrates, including, but not limited to companion animals and food animals. In particularly preferred embodiments, the mammal being treated is not concurrently suffering from an antibacterial infection.

The pharmaceutical compositions used in this invention may be prepared by combining the compounds of formula (I) with a solid or liquid pharmaceutically acceptable carrier and, optionally, with pharmaceutically acceptable adjuvants and excipients employing standard and conventional techniques. Solid form compositions include powders, tablets, dispersible granules, capsules, cachets and suppositories. A solid carrier can be at least one substance which may also function as a diluent, flavoring agent, solubilizer, lubricant, suspending agent, binder, tablet disintegrating agent, and encapsulating agent. Inert solid carriers include magnesium carbonate, magnesium stearate, talc, sugar, lactose, pectin, dextrin, starch, gelatin, cellulosic materials, low melting wax, cocoa butter, and the like. Liquid form compositions include solutions, suspensions and emulsions. For example, there may be provided solutions of the compounds used in this invention dissolved in water and water-propylene glycol and water-polyethylene glycol systems, optionally containing suitable conventional coloring agents, flavoring agents, stabilizers and thickening agents.

Preferably, the pharmaceutical composition is provided employing conventional techniques in unit dosage form containing effective or appropriate amounts of the active component.

The quantity of active component, that is the compound used according to this invention, in the pharmaceutical composition and unit dosage form thereof may be varied or adjusted widely depending upon the particular application, the potency of the particular compound, the desired concentration. Generally, the quantity of active component will range between 0.5% to 90% by weight of the composition.

In therapeutic use for treating or preventing osteoporosis, bone resorption or other bone diseases in vertebrate animals, the compounds or pharmaceutical compositions thereof will be administered orally, nasally, parenterally, topically, transdermally, or rectally at a dosage to obtain and maintain a concentration, that is, an amount, or blood-level of active component in the animal undergoing treatment which will be effective. Generally, such effective amount of dosage of active component will be in the range of about 0.1 to about 100, more preferably about 3.0 to about 50 mg/kg of patient body weight/day. It is to be understood that the dosages may vary depending upon the requirements of the patient, the severity of the condition being treated, and the particular compound being used. Also, it is to be understood that the initial dosage administered may be increased beyond the above upper level in order to rapidly achieve the desired blood-level or the initial dosage may be smaller than the optimum and the daily dosage may be progressively increased during the course of treatment depending on the particular situation. If desired, the daily dose may also be divided into multiple doses for administration, e.g., 2-4 four times per day. Once a day delivery of the compound using osmotic delivery technology such as the OROS system developed by Alza Corp. is also contemplated as falling within the scope of the invention.

When the compounds according to this invention are administered parenterally, i.e., by injection, for example, by intravenous injection or by other parenteral routes of administration, they will generally contain a pharmaceutically acceptable amount of the compound or a soluble salt (acid addition salt or base salt) dissolved in a pharmaceutically acceptable liquid carrier such as, for example, water-for-injection and a buffer to provide a suitably buffered isotonic solution, for example, having a pH of about 3.5-6. Suitable buffering agents include, for example, trisodium orthophosphate, sodium bicarbonate, sodium citrate, N-methylglucamine, L(+)-lysine and L(+)-arginine to name but a few representative buffering agents. The compound of this invention generally will be dissolved in the carrier in an amount sufficient to provide a pharmaceutically acceptable injectable concentration in the range of about 1 mg/mL to about 400 mg/mL of solution. The resulting liquid pharmaceutical composition will be administered so as to obtain the above-mentioned effective amount of dosage. The compounds according to this invention are advantageously administered orally in solid and liquid dosage forms.

The invention is described in greater detail by the following non-limiting examples.

### EXAMPLE 1

(S)-trans-[[3-[3-Fluoro-4-(tetrahydro-1-oxido-2H-thiopyran-4-yl)phenyl]-2-oxo-5-oxaczolidinyl]methyl]thiourea

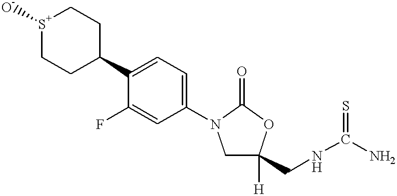

Step 1: A solution of 1,1′-thiocarbonyl-2(1H)-pyridone (192 mg, 0.827 mmol) in anhydrous methylene chloride (8.3 mL) at 0° C. under a nitrogen atmosphere was treated with a solution of (S)-trans-3-[3-fluoro-4-(tetrahydro-1-oxido-2H-thiopyran-4-yl)phenyl]-5-aninomethyl-2-oxazolidinone, as prepared in Example 9 of WO98/54161, Step 1, (225 mg, 0.689 mmol) in anhydrous methylene chloride (28 mL) over 30 minutes. The resulting mixture was stirred at 0° C. for 30 minutes and at ambient temperature for 40 minutes and was then diluted with methylene chloride (20 mL), washed with water (15 mL) and brine (15 mL), dried over anhydrous sodium sulfate and concentrated in vacuo. The crude product was chromatographed on silica gel (32-63 mm, 40 g), eluting with a gradient of acetonitriletmethylene chloride (30/70-60140) under 15 psi N₂, and those fractions with an R_{f}=0.12 by TLC (acetonitrile/methylene chloride, 30/70) were pooled and concentrated to give (S)-trans-3-[3-Fluoro-4-(tetrahydro-1-oxido-2H-thiopyran-4-yl)phenyl]-5-isothiocyanatomethyl-2-oxazolidinone, mp 165 -167° C.

Step 2: A solution of (S)-trans-3-[3-fluoro-4-(tetrahydro-1-oxido-2H-thiopyran-4-yl)phenyl]-5-isothiocyanatomethyl-2-oxazolidinone (Step 1, 230 mg, 0.624 mmol) in anhydrous tetrahydrofuran (31.2 mL) at 0° C. under a nitrogen atmosphere was treated (bubbled) with a stream of ammonia gas for 5 minutes. The reaction pot was sealed, and the resulting mixture was stirred at 0° C. for 1 hour. The excess ammonia was then removed under a stream of nitrogen, and the reaction mixture was concentrated in vacuo to give the crude product. Trituration with methanolmethylene chloride/diethyl ether gave the title compound, mp 209 -210° C. (dec.).

### EXAMPLE 2

(S)-N-[[3-[3-Fluoro-4-(4-thiomorpholinyl)phenyl] -2-oxo-5-oxazo lidinyl]methyl]thio-acetamide, thiomorpholine S-oxide (34).

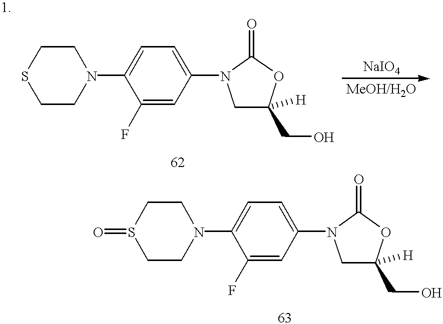

An ice cold, stirred mixture of sodium metaperiodate (1.08 g, 5.05 mmol) and water (12 mL), under nitrogen, was treated with 62 (1.5 g, 4.8 mmol) and MeOH (17 mL) and kept at 6° C. for 18 h and at 4° C. for 3 h. It was then treated with additional sodium metaperiodate (0.1 g), kept at 4° C. for 3 h and extracted with CHCl₃. The extract was dried (MgSO₄) and concntrated to give 1.4 g of 63: ¹H NMR [300 MHz, (CD₃)₂SO] d 2.84 (m, 2H), 3.01 (m, 2H), 3.16 (m, 2H), 3.50 (m, 3H), 3.65 (m, 1H), 3.77 (d,d, 1H), 4.03 (t, 1H), 4.66 (m, 1H), 5.18 (t, 1H), 7.16 (m, 2H), 7.52 (mn, 1H); MS(ES) m/z 329 (M+H⁺), 351 (M+Na⁺).

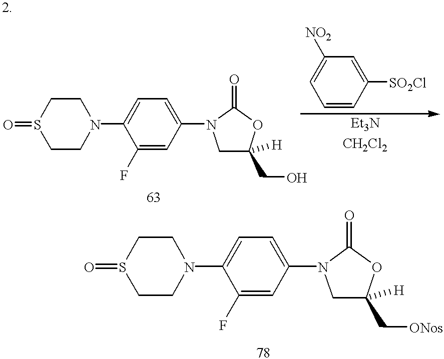

An ice cold, stirred mixture of 63 (1.27 g, 3.87 mmol) and triethylamine (0.732 mL, 5.25 mmol) in CH₂Cl₂ (130 mL), under nitrogen, was treated with m-nitrobenzenesulfonyl chloride (1.15 g, 5.19 mmol) and kept at ambient temperature for about 24 h. It was diluted with CH₂Cl₂, washed with water and brine, dried (Na₂SO₄) and concentrated to give 78 which was used in the next reaction without purification.

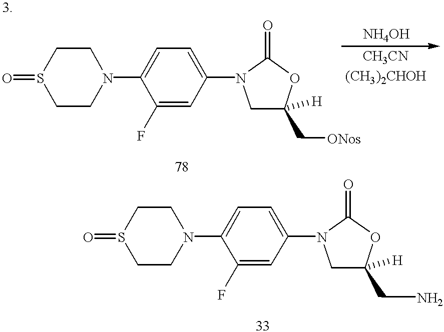

A stirred mixture ol the product (78) from the previous reaction, acetonitrile (70 mL) and isopropanol (70 mL) was treated with concentrated ammonium hydroxide (70 mL, 29.9% NH₃) and kept at 40° C. for 2 h, at ambient temperature for 18 h and at 40-45° C. for 4 h; it was concentrated to about 50 mL, diluted with water and extracted with CH₂Cl₂. The extracts were washed with water and brine, dried (MgSO₄) and concentrated. Chromatography of the residue on silica gel with 5% MeOH-CHCl₃ gave 0.58 g of 33: MS(ES) m/z 328 (M+H⁺), 350 (M+Na⁺); ¹H NMR [300 MHz, (CD₃)₂SO] d 2.81 (m, 4H), 3.01 (m, 2H), 3.16 (m, 2H), 3.30 (broad s), 3.49 (m, 2H), 3.80 (d,d, 1H), 4.01 (t, 1H), 4.58 (m, 1H), 7.19 (m, 2H), 7.51 (m, 1H).

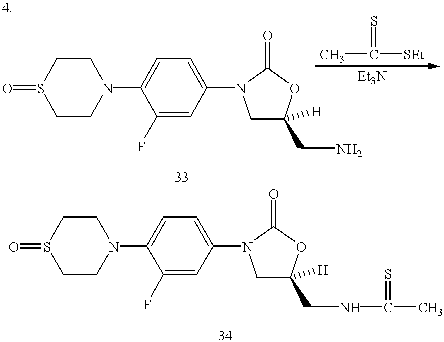

A stirred suspension of 33 (3.7 g, 0.011 mol) and triethylamine (3.5 mL, 0.025 mol) in THF (120 mL) was cooled, in an ice bath, under nitrogen, treated, dropwise during 2 min, with a solution of ethyl dithioacetate (1.47 mL, 0.0128 mol) in THF (2 mL) and kept at ambient temperature for 22 h. The resulting solution was concentrated and the residue crystallized from acetonitrile to give 3.61 g of 34: mp 176-177° C.; ¹H NMR [300 MHz, (CD₃)₂SO] d 2.42 (s, 3H), 2.85 (m, 2H), 3.01 (m, 2H), 3.18 (m, 3H), 3.50 (m, 2H), 3.78 (d,d, 1H), 3.89 (broad s, 2H), 4.12 (t, 1H), 4.92 (m, 1H), 7.18 (m, 2H), 7.49 (m, 1H), 10.33 (s, 1H); IR (DRIFT) 3186, 3102, 1741 cm⁻¹; MS(ES) m/z 386 (M+H⁺), 408 (M+Na⁺). Anal. calcd for C₁₆H₂₀FN₃O₃S₂°0.5 H₂O: C, 48.71; H, 5.37; N, 10.65; S, 16.26; H₂O, 2.38. Found: C, 48.75; H, 5.17; N, 10.72; S, 16.07; H₂O, 1.72.

### Use of the Compounds

The toxicity of the compound of Example 2 was evaluated in rats treated orally with 30, 60, 120, or 200 mg/kg/day of the compound of Example 2, water, or vehicle (containing 80% propylene glycol, 5% cremophor, and 30-mg/ml povidone) for 4 weeks given in 2 divided doses at 8 hours apart. Reversibility of drug-induced changes was determined 4 and 8 weeks following drug withdrawal. The pathogenesis of the bone changes was evaluated in a study in rats treated with 200 mg/kg/day for 3 or 7 days. TGF-betal protein levels in bone marrow supernatants and serum were determined using ELISA at the end of each of the dosing period. Toxicity was evaluated using clinical, hematological, biochemical, and pathologic end points.

All doses, but 200 mg/kg/day, were well tolerated clinically. In addition to anorexia, weight loss, GI effects, hematopoietic suppression and lymphoid depletion, administration of the compound of Example 2, at 120 and 200 mg/kg/day, caused an increase in bone mass and density (hyperostosis) of the trabecular (spongy) bone of the sternum, femur/tibia, vertebrae, and basal cranium. Endosteal cortical bone formation occurred rarely. Hyperostosis, which became evident by Day 7 was characterized by proliferation of osteoblasts and deposition of osteoid within the marrow cavity in a trabecular configuration and/or as deposits of new bone over the existing trabeculae. At Day 28, marrow cavities were traversed by abundant normal looking trabecular bone that was lined by some peri-trabecular osteoblast proliferation and osteoid deposition. There were no significant osseous changes on Day 3 except for some subtle focal proliferation/activation of osteoblasts. The newly formed bone spicules, still present after 4 and 8 weeks of drug withdrawal, were qualitatively indistinguishable from the pre-existing bone. In relatively severe cases, osteoblast proliferation and osteoid accumulation on Day 7 and newly formed trabecular bones on Day 28 or after the recovery periods compromised or obliterated the marrow spaces. However, the bone marrow did not show changes other than hypocellularity related to hematopoietic suppression. TGF-betal was modestly but significantly increased in marrow supernatants on Day 3 but not on Day 7 or 28. Serum TGF-beta I was slightly but significantly decreased on Day 3. Increased levels of TGF-betal in marrow supernatant preceded hyperostosis suggesting a possible mechanistic relationship. The compound of Example 2 increased bone mass and density within a short period ol time in an orderly manner at a well-tolerated doses. Newly formed bone that persisted for up to X weeks after drug withdrawal (the longest period evaluated) was morphologically indistinguishable from normal trabecular bones.

Having described the invention in detail and by reference to the preferred embodiments thereof, it will be apparent that modifications and variations are possible without departing from the scope of the appended claims.

What is claimed is:

1. A method of treating or preventing osteoporosis, bone resorption or other bone disease in a vertebrate mammal, comprising the step of administering to a mammal in need of such treatment, an effective amount to a compound of formula I 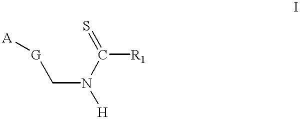
or pharmaceutical acceptable salts thereof wherein:
G is 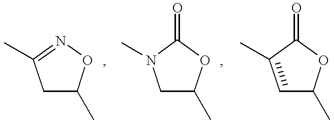
R₁ is
a) H,
b) NH₂,
c) NH—C₁₋₄ alkyl,
d) C₁₋₄ alkyl,
e) —OC₁₋₄ alkyl,
f) —S C₁₋₄ alkyl,
g) C₁₋₄ alkyl substituted with 1-3 F, 1-2 Cl, CN or —COOC₁₋₄ alkyl,
h) C₃₋₆ cycloalkyl,
i) N(C₁₋₄ alkyl)₂ or
j) N(CH₂)₂₋₅;
A is 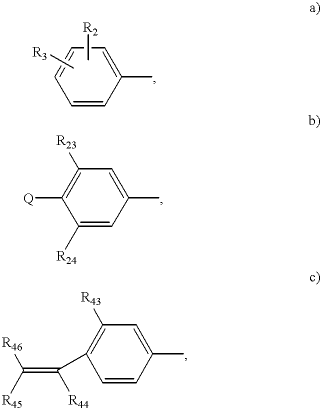
d) a 5-membered heteroaromatic moiety having one to three atoms selected from the group consisting of S, N, and O,
wherein the 5-membered heteroaromatic moiety is bonded via a carbon atom,
wherein the 5-membered heteroaromatic moiety can additionally have a fused-on benzene or naphthyl ring,
wherein the heteroaromatic moiety is optionally substituted with one to three R₄₈,
e) a 6-membered heteroaromatic moiety having at least one nitrogen atom,
wherein the heteroaromatic moiety is bonded via a carbon atom,
wherein the 6-membered heteroaromatic moiety can additionally have a fused-on benzene or naphthyl ring,
wherein the heteroaromatic moiety is optionally substituted with one to three R₅₅,
f) a β-carbolin-3-yl, or indolizinyl bonded via the 6-membered ring, optionally substituted with one to three R₅₅, 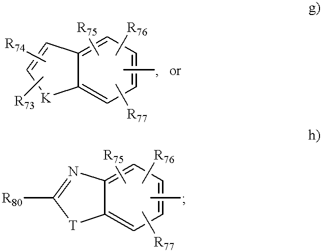
wherein R₂ is
a) H,
b) F,
c) Cl,
d) Br,
e) C₁₋₃ alkyl,
f) NO₂, or
g) R₂ and R₃ taken together are —O—(CH₂)ₕ—O—;
R₃ is
a) —S(═O)ᵢR₄,
b) —S(═O)₂—N═S(O)ⱼR₅R₆,
c) —SC(═O)R₇,
d) —C(═O)R₈,
e) —C(═O)R₉,
f) —C(═O)NR₁₀R₁₁,
g) —C(═NR₁₂)R₈,
h) —C(R₈)(R₁₁)—OR₁₃,
i) —C(R₉)(R₁₁)—OR₁₃,
j) —C(R₈)(R₁₁)—OC)═O)R₁₃,
k) —C(R₉)(R₁₁)—OC(═O)R₁₃,
l) —NR₁₀R₁₁,
m) —N(R₁₀)—C(═O)R₇,
n) —N(R₁₀)—S(═O)ᵢR₇,
o) —C(OR₁₄)(OR₁₅)R₈,
p) —C(R₈)(R₁₆)—NR₁₀R₁₁, or
q) C₁₋₈ alkyl substituted with one or more ═O other than at alpha position, —S(═O)ᵢR₁₇, —NR₁₀R₁₁, C₂₋₅ alkenyl, or C₂₋₅ alkynyl;
R₄ is
a) C₁₋₄ alkyl optionally substituted with one or more halos, OH, CN, NR₁₀R₁₁, or —CO₂R₁₃,
b) C₂₋₄ alkenyl,
c) —NR₁₆R₁₈,
d) —N₃,
e) —NHC(═O)R₇,
f) —NR₂₀C(═O)R₇,
g) —N(R₁₉)₂,
h) —NR₁₆R₁₉, or
i) —NR₁₉R₂₀,
R₅ and R₆ at each occurrence are the same or different and are
a) C₁₋₂ alkyl, or
b) R₅ and R₆ taken together are —(CH₂)ₖ—;
R₇ is C₁₋₄ alkyl optionally substituted with one or more halos;
R₈ is
a) H, or
b) C₁₋₈ alkyl optionally substituted with one or more halos, or C₃₋₈ cycloalkyl;
R₉ C₁₋₄ alkyl substituted with one or more
a) —S(═O)R₁₇,
b) —OR₁₃,
c) —OC(═O)R₁₃,
d) —NR₁₀R₁₁, or
e) C₁₋₅ alkenyl optionally substituted with CHO;
R₁₀ and R₁₁ at each occurrence are the same or different and are
a) H,
b) C₁₋₄ alkyl, or
c) C₁₋₄ cycloapyl;
R₁₂ is
a) —NR₁₀R₁₁,
b) —OR₁₀; or
c) —NHC(═O)R₁₀;
R₁₃ is
a) H, or
b) C₁₋₄ alkyl;
R₁₄ and R₁₅ at each occurrence are the same or different and are
a) C₁₋₄ alkyl, or
b) R₁₄ and R₁₅ taken together are —(CH)_{I}—;
R₁₆ is
a) H,
b) C₁₋₄ alkyl, or
c) C₃₋₈ cycloalkyl;
R₁₇ is
a) C₁₋₄ alkyl, or
b) C₃₋₈ cycloalkyl;
R₁₈ is
a) H,
b) C₁₋₄ alkyl,
c) C₂₋₄ alkenyl,
d) C₃₋₄ cycloalkyl,
e) —OR₁₃ or
f) —NR₂₁R₂₂;
R₁₉ is
a) Cl,
b) Br, or
c) I;
R₂₀ is a physiologically acceptable cation;
R₂₁ and R₂₂ at each occurrence are the same or different and are
a) H,
b) C₁₋₄ alkyl, or
c) —NR₂₁R₂₂ taken together are —(CH₂)ₘ—;
wherein R₂₃ and R₂₄ at each occurrence are the same or different and are
a) H,
b) F,
c) Cl,
d) C₁₋₂ alkyl,
e) CN
f) OH,
g) C₁₋₂ alkoxy,
h) nitro, or
i) amino;
Q is 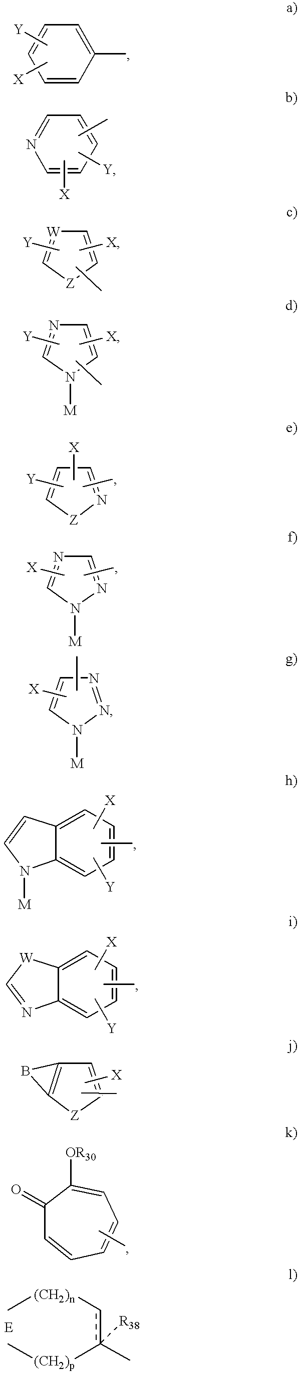
m) a diazinyl group optionally substituted with X and Y,
n) a triazinyl group optionally substituted with X and Y,
o) a quinolinyl group optionally substituted with X and Y,
p) a quinoxalinyl group optionally substituted with X and Y,
q) a naphthyridinyl group optionally substituted with X and Y, 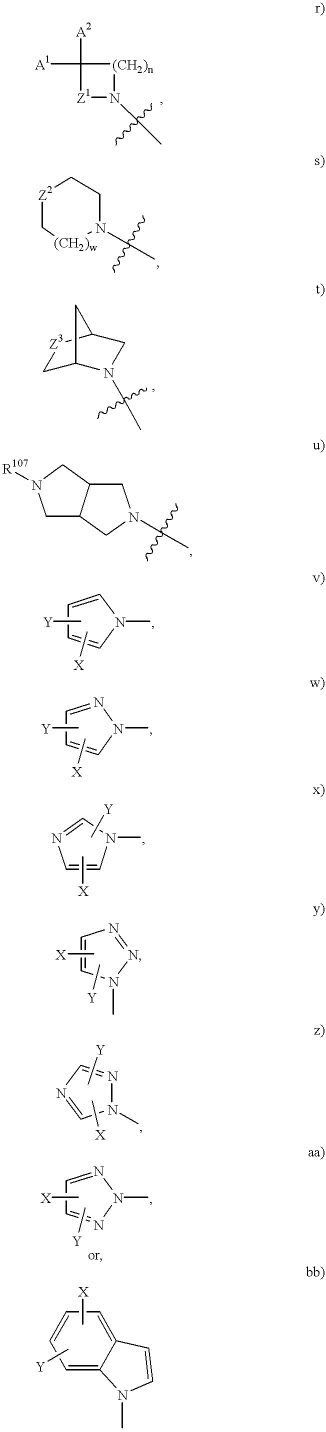
Q and R₂₄ taken together are 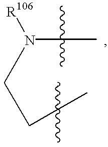
wherein Z¹ is
a) —CH₂—,
b) —CH(R¹⁰⁴)—CH₂—,
c) —C(O)—, or
d) —CH₂CH₂CH₂—;
wherein Z² is
a) —O₂S—,
b) —O—,
c) —N(R¹⁰⁷)—,
d) —OS—, or
e) —S—;
wherein Z³ is
a) —O₂S—,
b) —O—,
c) —OS—, or
d) —S—;
wherein A¹ is
a) H—, or
b) CH₃;
wherein A² is
a) H—,
b) HO—,
c) CH₃—,
d) CH₃O—,
e) R¹⁰²O—CH₂—C(O)—NH—
f) R¹⁰³O—C(O)—NH—,
g) (C₁-C₂)alkyl-O—C(O)—,
h) HO—CH₂—,
i) CH₃O—NH—,
j) (C₁-C₃)alkyl-O₂C—
k) CH₃—C(O)—,
l) CH₃—C(O)—CH₂—, 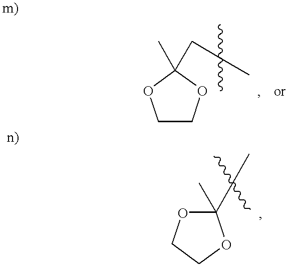
A¹ and A² taken together are: 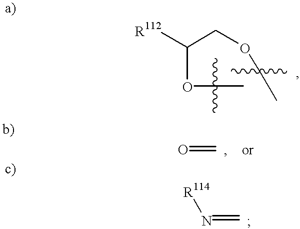
wherein R¹⁰² is
a) H—,
b) CH₂—,
c) phenyl-CH₂—, or
d) CH₃C(O);
wherein R¹⁰³ is
a) (C₁-C₃)alkyl-, or
b) phenyl-;
wherein R¹⁰⁴ is
a) H—, or
b) HO—;
wherein R¹⁰⁵ is
a) H—,
b) (C₁-C₃)alkyl-,
c) CH₂=CH—CH₂—, or
d) CH₃—O—(CH₂)₂—;
wherein R¹⁰⁶ is
a) CH₃—C(O)—,
b) H—C(O)—,
c) Cl₂CH—C(O)—,
d) HOCH₂—C(O)—,
e) CH₃SO₂—, 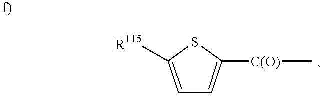
g) F₂CHC(O)—, 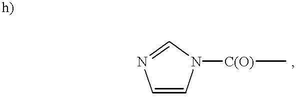
i) H₃C—C(O)—O—CH₂—C(O)—,
j) H—C(O)—O—CH₂—C(O)—, 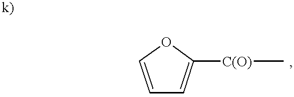
l ) HC≡CH—CH₂O—CH₂—C(O)—, or
m) phenyl—C₂-O—CH₂—C(O)—;
wherein R¹⁰⁷ is
a) R¹⁰²O—C(R¹¹⁰)(R¹¹¹)—C(O)—,
b) R¹⁰³O—C(O)—,
c) R¹⁰⁸—C(O)—, 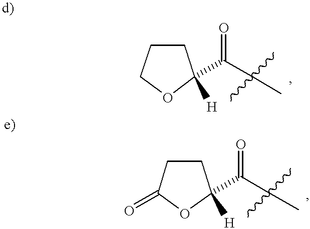
f) H₃C—C(O)—(CH₂)₂—C(O)—,
g) R¹⁰⁹—SO₂—, 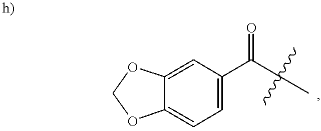
i) HO—CH₂—C(O)—,
j) R¹¹⁶—(CH₂)₂—,
k) R¹¹³—C(O)—O—CH₂—C(O)—,
l) (CH₃)₂N—CH₂—C(O)—NH—,
m) NC—CH₂—,
n) F₂—CH—CH₂—, or
o) R¹⁵⁰R¹⁵¹NSO₂
wherein R¹⁰⁸ is
a) H—,
b) (C₁-C₄)alkyl,
c) aryl —(CH₂)ₚ,
d) ClH₂C—,
e) Cl₂HC—,
f) FH₂C—,
g) F₂HC—,
h) (C₃-C₆)cycloalkyl, or
i) CNCH₂—,
wherein R¹⁰⁹ is
a) alkylC₁-C₄,
b) —CH₂Cl
c) —CH₂CH═CH₂,
d) aryl, or
e) —CH₂CN;
wherein R¹¹⁰ and R¹¹¹ are independently
a) H—,
b) CH₃—; or
wherein R¹¹² is
a) H—,
b) CH₃O—CH₂O—CH₂—, or
c) HOCH₂—;
wherein R¹¹³ is
a) CH₃—,
b) HOCH₂—,
c) (CH₃)₂N-phenyl, or
d) (CH₃)₂N—CH₂—;
wherein R¹¹⁴ is
a) HO—,
b) CH₃O—,
c) H₂N—,
d) CH₂O—C(O)—O—,
e) CH₃—C(O)—O—CH₂—C(O)—O—,
f) phenyl-CH₂—O—CH₂—C(O)—O—,
g) HO—(CH₂)₂—O—,
h) CH₃O—CH₂—O—(CH₂)₂—O—, or
i) CH₃O—CH₂—O—;
wherein R¹¹³ is
a) CH₃—,
b) HOCH₂—,
c) (CH₃)₂N-phenyl, or
d) (CH₃)₂N—CH₂—;
wherein R¹¹⁶ is
a) H—, or
b) Cl—;
wherein R¹¹⁶ is
a) HO—
b) CH₃O—, or
c) F;
wherein R¹⁵⁰ and R¹⁵¹ are each H or alkyl C₁-C₄ or R¹⁵⁰ and R¹⁵¹ taken together with the nitrogen atom to which each is attached form a monocyclic heterocyclic ring having from 3 to 6 carbon atoms;
B is an unsaturated 4-atom linker having one nitrogen and three carbons;
M is
a) H,
b) C₁₋₈ alkyl,
c) C₃₋₈ cycloalkyl,
d) —(CH₂)ₘOR₁₃, or
e) —(CH₂)ₕ—NR₂₁R₂₂;
Z is
a) O,
b) S, or
c) NM;
W is
a) CH,
b) N, or
c) S or O when Z is NM;
Y is
a) H,
b) F,
c) Cl,
d) Br,
e) C₁₋₃ alkyl, or
f) NO₂;
X is
a) H,
b) —CN,
c) OR₂₇,
d) halo,
e) NO₂,
f) tetrazoyl,
g) —SH,
h) —S(═O)ᵢR₄,
i) —S(═O)₂—N═S(O)ⱼR₅R₆,
j) —SC(═O)R₇,
k) —C(═O)R₂₅,
l) —C(═O)NR₂₇R₂₈,
m) —C(═NR₂₉)R₂₅,
n) —C(R₂₅)(R₂₈)—OR₁₃,
o) —C(R₂₅)(R₂₈)—OR₁₃,
p) —C(R₂₈)(OR₁₃)—(CH₂)ₙ—NR₂₇R₂₈,
q) —NR₂₇R₂₈,
r) —N(R₂₇)C(═O)R₇,
s) —N(R₂₇)—S(═O)ᵢR₇,
t) —C(OR₁₄)(OR₁₅)R₂₈,
u) —C(R₂₅)(R₁₆)—NR₂₇R₂₆, or
v) C₁₋₈ alkyl substituted with one or more halos, OH, ═O other than at alpha position, —S(═O)ᵢR₁₇, —NR₂₇R₂₈, C₂₋₅ alkenyl, C₂₋₅ alkynyl, or C₃₋₈ cycloalkyl;
R₄, R₅, R₆, R₇, R₁₃, R₁₄, R₁₅, R₁₆, and R₁₇ are the same as defined above;
R₂₅ is
a) H,
b) C₁₋₅ alkyl optionally substituted with one or more halos, C₃₋₅ cycloalkyl, C₁₋₄ alkyl substituted with one or more of —S(═O)ᵢR₁₇, —OR₁₈, or OC(═O)R₁₈, NR₂₇R₂₈, or
c) C₂₋₅ alkenyl optionally substituted with CHO, or CO₂R₁₃;
R₂₆ is
a) R₂₈, or
b) NR₂₇N₂₈;
R₂₇ and R₂₈ at each occurrence are the same or different and are
a) H,
b) C₁₋₈ alkyl,
c) C₃₋₈ cycloalkyl,
d) —(CH₂)ₘOR₁₃,
e) —(CH₂)ₕ—NR₂₁R₂₂, or
f) R₂₇ and R₂₈ taken together are —(CH₂)₂O(CH₂)₂—, —(CH₂)ₕCH(COR₇)—, or —(CH₂)₂N(CH₂)₂(R₇);
R₂₉ is
a) —NR₂₇R₂₈,
b) —OR₂₇, or
c) —NHC(═O)R₂₈;
wherein R₃₀ is
a) H,
b) C₁₋₈ alkyl optionally substituted with one or more halos, or
c) C₁₋₈ alkyl optionally substituted with one or more OH, or C₁₋₆ alkoxy,
wherein E is
a) N
b) —S(═O)ᵢ, or
c) O;
R₃₈ is
a) H,
b) C₁₋₆ alkyl,
c) —(CH₂)_{q}-aryl, or
d) halo;
R₃₉ is
a) H,
b) C₁₋₆ alkyl optionally substituted with one or more OH, halo, or —CN,
c) —(CH₂)_{q}-aryl,
d) —CO₂R₄₀,
e) —COR₄₁,
f) —C(═O)—(CH₂)_{q}—C(═O)R₄₀,
g) —S(═O)₂—C₁₋₆ alkyl,
h) —S(═O)₂—(CH₂)_{q}-aryl, or
i) —(C═O)ⱼ—Het;
R₄₀ is
a) H,
b) C₁₋₆ alkyl optionally substituted with one or more OH, halo, or —CN,
c) —(CH₂)_{q}-aryl, or
d) —(CH₂)_{q}—OR₄₂;
R₄₁ is
a) C₁₋₆ alkyl optionally substituted with one or more OH, halo, or —CN,
b) —(CH₂)_{q}-aryl, or
c) —(CH₂)_{q}—R₄₂;
R₄₂ is
a) H,
b) C₁₋₆ alkyl
c) —(CH₂)_{q}-aryl, or
d) —C(═O)—C₁₋₆ alkyl;
aryl is
a) phenyl,
b) pyridyl, or
c) napthyl; a to c optionally substituted with one or more halo, —CN, OH, SH, C₁₋₆ alkyl, C₁₋₆ alkoxy, or C₁₋₆ alkylthio;
wherein R₄₃ is
a) H,
b) C₁₋₂ alkyl,
c) F, or
d) OH;
R₄₄ is
a) H,
b) CF₃,
c) C₁₋₃ alkyl optionally substituted with one or more halo,
d) phenyl optionally substituted with one or more halo,
e) R₄₄ and R₄₅ taken together are a 5-, 6-, or 7-membered ring of the formula, or 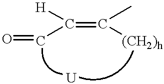
f) R₄₄ and R₄₅ taken together are —(CH₂)ₖ—, when R₄₆ is an electron-withdrawing group;
R₄₅ and R₄₆ at each occurrence are the same or different and are
a) an electron-withdrawing group,
b) H,
c) CF₃,
d) C₁₋₃ alkyl optionally substituted with one halo,
e) phenyl, provided at least one of R₄₅ or R₄₆ is an electron-withdrawing group, or
f) R₄₅ and R₄₆ taken together are a 5-, 6-, 7-membered ring of the formula 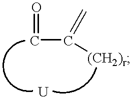
U is
a) CH₂,
b) O,
c) S, or
d) NR₄₇;
R₄₇ is
a) H, or
b) C₁₋₅ alkyl;
wherein R₄₈ is
a) carboxyl,
b) halo,
c) —CN,
d) mercapto,
e) formyl,
f) CF₃,
g) —NO₂,
h) C₁₋₆ alkoxy,
i) C₁₋₆ alkoxycarbonyl,
j) C₁₋₆ alkythio,
k) C₁₋₆ acyl,
l) —NR₄₉R₅₀,
m) C₁₋₆ alkyl optionally substituted with OH, C₁₋₅ alkoxy, C₁₋₅ acyl, or —NR₄₉R₅₀,
n) C₂₋₈ alkenylphenyl optionally substituted with one or two R₅₁,
o) phenyl optionally substituted with one or two R₅₁,
p) a 5-, or 6-membered (un)saturated heterocyclic moiety having one to three atoms selected from the group consisting of S, N, and O, optionally substituted with one or two R₅₁, or 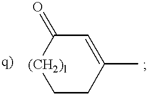
R₄₉ and R₅₀ at each occurrence are the same or different and are
a) H,
b) C₁₋₄ alkyl,
c) C₅₋₆ cycloalkyl, or
d) R₄₉ and R₅₀ taken together with the nitrogen atom is a 5-, 6-membered saturated heterocyclic moiety which optionally has a further hetero atom selected from the group consisting of S, N, and O, and can in turn be optionally substituted with, including on the further nitrogen atom, C₁₋₃ alkyl, or C₁₋₃ acyl;
R₅₁ is
a) carboxyl,
b) halo,
c) —CN,
d) mercapto,
e) formyl,
f) CF₃,
g) —NO₂,
h) C₁₋₆ alkoxy,
i) C₁₋₆ alkoxycarbonyl,
j) C₁₋₆ alkythio,
k) C₁₋₆ acyl,
l) C₁₋₆ alkyl optionally substituted with OH, C₁₋₆ alkoxy, C₁₋₆ acyl, or —NR₄₉R₅₀,
m) phenyl,
n) —C(═O)NR₅₂R₅₃,
o) —NR₄₉R₅₀,
p) —N(R₅₂)(—SO₂R₅₄),
q) —SO₂—NR₅₂R₅₃, or
r) —S(═O)ᵢR₅₄;
R₅₂ and R₅₃ at each occurrence are the same or different and are
a) H,
b) C₁₋₆ alkyl, or
c) phenyl;
R₅₄ is
a) C₁₋₄ alkyl, or
b) phenyl optionally substituted with C₁₋₄ alkyl;
wherein R₅₅ is
a) carboxyl,
b) halo,
c) —CN,
d) mercapto,
e) formyl,
f) CF₃,
g) —NO₂,
h) C₁₋₆ alkoxy,
i) C₁₋₆ alkoxycarbonyl,
j) C₁₋₆ alkythio
k) C₁₋₆ acyl,
l) —NR₅₆R₅₇,
m) C₁₋₆ alkyl optionally substituted with OH, C₁₋₅ alkoxy, C₁₋₅ acyl, or
n) C₂₋₈ alkenylphenyl optionally substituted with one or two R₅₈,
o) phenyl optionally substituted with one or two R₅₈,
p) a 5- or 6-membered (un)saturated heterocyclic moiety having one to three atoms selected from the group consisting of S, N, and O, optionally substituted with one or two R₅₈, or 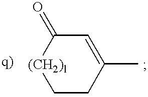
R₅₆ and R₅₇ at each occurrence are the same or different and are
a) H,
b) formyl,
c) C₁₋₄ alkyl,
d) C₁₋₄ acyl,
e) phenyl,
f) C₃₋₆ cycloalkyl, or
g) R₅₆ and R₅₇ taken together with the nitrogen atom is a 5-, 6-membered saturated heterocyclic moiety which optionally has a further hetero atom selected from the group consisting of S, N, and O, and can in turn be optionally substituted with, including on the further nitrogen atom, phenyl, pyrimidyl, C₁₋₃ alkyl, or C₁₋₃ acyl;
R₅₈ is
a) carboxyl,
b) halo,
c) —CN,
d) mercapto,
e) formyl,
f) CF₃,
g) —NO₂,
h) C₁₋₆ alkoxy,
i) C₁₋₆ alkoxycarbonyl,
j) C₁₋₆ alkythio,
k) C₁₋₆ acyl,
l) phenyl,
m) C₁₋₆ alkyl optionally substituted with OH, azido, C₁₋₅ alkoxy, C₁₋₅ acyl, —NR₆₅R₆₆., —SR₆₇, —O—SO₂R₆₈, or 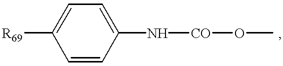
n) —C(═O)NR₅₉R₆₀,
o) —NR₅₆R₅₇,
p) —N(R₅₉)(—SO₂R₅₄),
q) —SO₂—NR₅₉R₆₀,
r) —S(═O)ᵢR₆₄,
s) —CH═N—R₆₁, or
t) —CH(OH)—SO₃R₆₄;
R₅₄ is the same as defined above;
R₅₉ and R₆₀ at each occurrence are the same or different and are
a) H,
b) C₁₋₆alkyl,
c) phenyl, or
d) tolyl;
R₆₁ is
a) OH,
b) benzyloxy,
c) —NH—C(═O)—NH₂,
d) —NH—C(═S)—NH₂, or
e) —NH—C(═NH)—NR₆₂R₆₃;
R₆₂ and R₆₃ at each occurrence are the same or different and are
a) H, or
b) C₁₋₄ alkyl optionally substituted with phenyl or pyridyl;
R₆₄ is
a) H, or
b) a sodiumion;
R₆₅ and R₆₆ at each occurrence are the same or different and are
a) H,
b) formyl,
c) C₁₋₄ alkyl,
d) C₁₋₄ acyl,
e) phenyl,
f) C₃₋₆ cycloalkyl,
g) R₆₅ and R₆₆ taken together are a 5-, 6-membered saturated heterocyclic moiety having one to three atoms selected from the group consisting of S, N, and O, optionally substituted with, including on the nitrogen atom, phenyl, pyrimidyl, C₁₋₃ alkyl, or C₁₋₃ acyl,
h) —P(O)(OR₇₀)(OR₇₁), or
i) —SO₂—R₇₂;
R₆₇ is 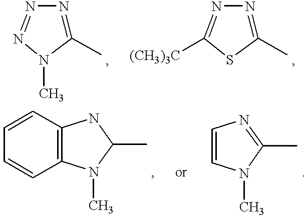
R₆₈ is C₁₋₃ alkyl;
R₆₉ is
a) C₁₋₆ alkoxycarbonyl, or
b) carboxyl;
R₇₀ and R₇₁ at each occurrence are the same or different and are
a) H, or
b) C₁₋₃ alkyl;
R₇₂ is
a) methyl,
b) phenyl, or
c) tolyl;
wherein K is
a) O, or
b) S;
R₇₃, R₇₄, R₇₅, R₇₆, and R₇₇ at each occurrence are the same or different and are
a) H,
b) carboxyl,
c) halo,
d) —CN,
e) mercapto,
f) formyl,
g) CF₃,
h) —NO₂,
i) C₁₋₆ alkoxy,
j) C₁₋₆ alkoxycarbonyl,
k) C₁₋₆ alkythio,
l) C₁₋₆ acyl,
m) —NR₇₈R₇₉,
n) C₁₋₆ alkyl optionally substituted with OH, C₁₋₅ alkoxy, C₁₋₅ acyl, —NR₇₈R₇₉, —N(phenyl)(CH₂—CH₂—OH), —O—CH(CH₃)(OCH₂CH₃), or —O-phenyl-[para-NHC(═O)CH₃],
o) C₂₋₈ alkenylphenyl optionally substituted with R₅₁,
p) phenyl optionally substituted with R₅₁, or
q) a 5-, or 6-membered (un)saturated heterocyclic moiety having one to three atoms selected from the group consisting of S, N, and O, optionally substituted with R₅₁;
R₅₁ is the same as defined above;
R₇₈ and R₇₉ at each occurrence are the same or different and are
a) H,
b) C₁₋₄ alkyl,
C) phenyl, or
d) R₇₈ and R₇₉ taken together with the nitrogen atom is a 5-, 6-membered saturated heterocyclic moiety which optionally has a further hetero atom selected from the group consisting of S, N, and O, and can in turn be optionally substituted with, including on the further nitrogen atom, C₁₋₃ alkyl or C₁₋₃ acyl;
wherein T is
a) O,
b) S, or
c) SO₂;
R₇₅, R₇₆, and R₇₇ are the same as defined above;
R₈₀ is
a) H,
b) formyl,
c) carboxyl,
d) C₁₋₆ alkoxycarbonyl,
e) C₁₋₈ alkyl,
f) C₂₋₈ alkenyl,
wherein the substituents (e) and (f) can be optionally substituted with OH, halo, C₁₋₆ alkoxy, C₁₋₆ acyl, C₁₋₆ alkylthio or C₋₁₆ alkoxycarbonyl, or phenyl optionally substituted with halo,
g) an aromatic moiety having 6 to 10 carbon atoms optionally substituted with carboxyl, halo, —CN, formyl, CF₃, —NO₂, C₁₋₆ alkyl, C₁₋₆ alkoxy, C₁₋₆ acyl, C₁₋₆ alkylthio, or C₁₋₆ alkoxycarbonyl;
h) —NR₈₁R₈₂,
i) —OR₉₀,
j) —S(═O)ᵢ—R₉₁,
k) —SO₂—N(R₉₂)(R₉₃), or
l) a radical of the following formulas:
R₈₁ and R₈₂ at each occurrence are the same or different and are
a) H,
b) C₃₋₆ cycloalkyl,
c) phenyl,
d) C₁₋₆ acyl,
e) C₁₋₈ alkyl optionally substituted with OH, C₁₋₆ alkoxy which can be substituted with OH, a 5-, or 6-membered aromatic heterocyclic moiety having one to three atoms selected from the group consisting of S, N, and O, phenyl optionally substituted with OH, CF₃, halo, —NO₂, C₁₋₄ alkoxy, —NR₈₃R₈₄, or 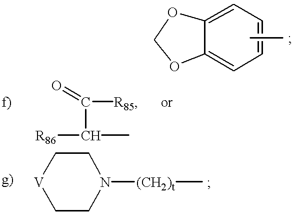
V is
a) O,
b) CH₂, or
c) NR₈₇;
R₈₃ and R₈₄ at each occurrence are the same or different and are
a) H, or
b) C₁₋₄ alkyl;
R₈₅ is
a) OH,
b) C₁₋₄ alkoxy, or
c) —NR₈₈R₈₉;
R₈₆ is
a) H, or
b) C₁₋₇ alkyl optionally substituted with indolyl, OH, mercaptyl, imidazoly, methylthio, amino, phenyl optionally substituted with OH, —C(═O)—NH₂, —CO₂H, or —C(═NH)—NH₂;
R₈₇ is
a) H,
b) phenyl, or
c) C₁₋₆ alkyl optionally substituted by OH;
R₈₈ and R₈₉ at each occurrence are the same or different and are
a) H,
b) C₁₋₅ alkyl
c) C₃₋₆ cycloalky, or
d) phenyl;
R₉₀ is
a) C₁₋₈ alkyl optionally substituted with C₁₋₆ alkoxy or C₁₋₆ hydroxy, C₃₋₆ cycloalkyl, a 6-membered aromatic optionally benzo-fised heterocyclic moiety having one to three nitrogen atoms, which can in turn be substituted with one or two —NO₂, CF₃, halo, —CN, OH, C₁₋₅ alkyl, C₁₋₅ alkoxy, or C₁₋₅ acyl; 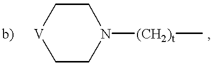
c) phenyl, or
d) pyridyl;
R₉₁ is
a) C₁₋₁₆ alkyl,
b) C₂₋₁₆ alkenyl,
wherein the substituents (a) and (b) can be optionally substituted with C₁₋₆ alkoxycarbonyl, or a 5-, 6-, 7-membered aromatic heterocyclic moiety having one to three atoms selected from the group consisting of S, N, and O,
c) an aromatic moiety having 6 to 10 carbon atoms, or
d) a 5-, 6-, 7-membered aromatic heterocyclic moiety having one to three atoms selected from the group consisting of S, N, and O,
wherein the substituents (c) and (d) can be optionally substituted with carboxyl, halo, —CN, formyl, CF₃, —NO₂, C₁₋₆ alkyl, C₁₋₆ alkoxy, C₁₋₆ acyl, C₁₋₆ alkylthio, or C₁₋₆ alkoxycarbonyl;
R₉₂ and R₉₃ at each occurrence are the same or different and are
a) H,
b) phenyl,
c) C₁₋₆ alkyl, or
d) benzyl;
R₉₄ and R₉₅ at each occurrence are the same or different and are
a) H,
b) OH,
c) C₁₋₆ alkyl optionally substituted with —NR₈₃R₈₄, or
d) R₉₄ and R₉₅ taken together are ═O;
R₉₆ is
a) an aromatic moiety having 6 to 10 carbon atoms,
b) a 5- or 6membered aromatic optionally benzo-fised heterocyclic moiety having one to three atoms selected from the group consisting of S, N, and O,
wherein the substituents (a) and (b) which can in turn be substituted with one or three —NO₂, CF₃, halo, —CN, OH, phenyl, C₁₋₅ alkyl, C₁₋₅ alkoxy, or C₁₋₅ acyl,
c) morpholinyl,
d) OH,
e) C₁₋₆ alkoxy,
f) —NR₈₃R₈₄,
g) —C(═O)—R₉₇, or
R₉₇ is 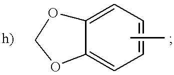
a) morpholinyl,
b) OH, or
c) C₁₋₆ alkoxy;
h is 1, 2, or 3;
i is 0, 1, or 2;
j is 0 or 1;
k is 3, 4,or 5;
l is 2 or 3;
m is 4 or 5;
n is 0, 1, 2, 3, 4, or 5;
p is 0, 1, 2, 3, 4, or 5; with the proviso that n and p together are 1, 2, 3, 4, or 5;
q is 1, 2, 3,or 4;
r is 2, 3, or 4;
t is 0, 1, 2, 3, 4, 5, or 6;
u is 1 or 2;
w is 0, 1, 2, or 3.

2. The method according to claim 1 wherein said mammal is a human.

3. The method according to claim 1 wherein the compound is administered in the range of about 0.1 to about 100 mg/kg of mammal body weight/day.

4. The method according to claim 1 wherein the compound is administered orally, nasally, parenterally, topically, transdermally, or rectally.

5. The method according to claim 1 wherein said compound is selected from the group consisting of:
(S)-trans-[[3-[3-Fluoro4(tetrahydro-1-oxido-2H-thiopyrantyl)phenyl]-2-oxo-5-oxazolidinyl]methyl]thiourea; and
(S)-N-[[3-[3-Fluoro-4-(4-thomorpholinyl)phenyl]-2-oxo-5-oxazohdinyl]methyl]thoacetamide, thiomorpholine S-oxide; and
pharmaceutically acceptable salts thereof.

6. The method according to claim 1 wherein said mammal is not suffering from an antibacterial infection.

7. A method of treating or preventing osteoporosis, bone resorption or other bone disease in a vertebrate mammal, comprising the step of administering to a mammal in need of such treatment, an effective amount of a compound of formula II 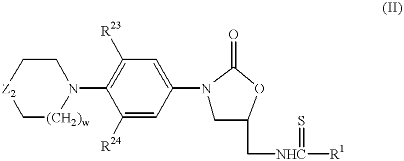
wherein Z₂ is —O₂S—, —O—, —N(R¹⁰⁷)—, —OS—, or —S—;
w is 0, 1, 2, or 3;
R²³ and R²⁴ are the same or different and can be H or F; and
R¹ is H, NH₂, NHalkylC₁-C₄; N(alkylC₁-C₄)₂; 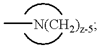
alkylC₁-C₄; OalkylC₁-C₄; SalkylC₁-C₄; alkylC₁-C₄ substituted with 1-3F, 1-2Cl, CN, or —COOalkylC₁-C₄, or cycloalkylC₃-C₆, wherein in each occurrence of the alkyl group may be straight or branched; and
R¹⁰⁷ is
a) R¹⁰²I—C(R¹¹⁰)(R¹¹¹)—C(O)—,
b) R¹⁰³O—C(O)—,
c) R¹⁰⁸—C(O)—,
d) R¹⁰⁹—SO₂—,
e) NC—CH₂—,
f) FCHCH₂—, or
g) R¹⁵⁰R¹⁵¹NSO₂;
wherein R¹⁰² is H, CH₃—, phenyl-CH₂—, or CH₃C(O); each of R¹¹⁰ and R¹¹¹ is selected from H or CH₃; R¹⁰³ is alkylC₁-C₃ or phenyl; R¹⁰⁸ is H, alkylC₁-C₄, aryl(CH₂)_{0.5}, CNCH₂—, ClCH₂—, Cl₂HC—, FH₂C—, F₂HC—, or cycloalkylC₁-C₆; R¹⁵⁰ and R¹⁵¹ are the same or different and arc selected from H, alkylC₁-C₄, or R¹⁵⁰ and R¹⁵¹ taken together with the nitrogen to which each is attached forms a monocyclic heterocyclic ring having from 3 to 6 carbon atoms.

8. The method according to claim 7 wherein said mammal is a human.

9. The method according to claim 7 wherein the compound is administered in the range of about 0.1 to about 100 mg/kg of mammal body weight/day.

10. The method according to claim 7 wherein the compound is administered orally, nasally, parenterally, topically, transdermally, or rectally.

11. The method according to claim 7 wherein said compound is selected from the group consisting of:
(S)-trans-[[3-[3-Fluorot(tetrahydro-1-oxido-2H-thopyran4fiyl)phenyl]-2-oxo-5-oxazolidinyl]methyl]thiourea; and
(S)-N-[[3-[3-Fluoro4(4thiomorpholinyl)phenyl]-2-oxo-5-oxazolidinyl]methyl]thio-acetamide, thiomorpholine S-oxide; and
pharmaceutically acceptable salts thereof.

12. The method according to claim 7 wherein said mammal is not suffering from an antibacterial infection.

13. The use of a compound of formula (I) or formula (II) to prepare a medicament for treating or preventing osteoporosis, bone resorption or other bone disease in a mammal.
